(19) United States
(12) Patent Application Publication
(10) Pub. No.: US 2002/0058244 A1
(43) Pub. Date: May 16, 2002

(54) Method for detecting uracil biosynthesis inhibitors and their use as herbicides

(76) Inventors: Marianne K. Pedersen, Princeton Junction, NJ (US); Iwona T. Birk, Raleigh, NC (US); Ann B. Orth, Langhorne, PA (US); Bijay K. Singh, Apex, NC (US); Berhane Tecle, Lawrenceville, NJ (US); Venkataraman Kameswaran, Pennington, NJ (US); Stephen S. Szucs, Lawrenceville, NJ (US)

Correspondence Address: MERCHANT & GOULD PC, P.O. BOX 2903, MINNEAPOLIS, MN, 55402-0903, US

(21) Appl. No.: 09/905,638

(22) Filed: Jul. 13, 2001

Related U.S. Application Data
(60) Provisional application No. 60/218,193, filed on Jul. 14, 2000.

(51) Int. Cl. C12Q 1/00; A01N 43/56; C07D 231/20

(52) U.S. Cl. 435/4; 504/116.1; 504/282; 548/370.1

(57) ABSTRACT

This invention relates to a method for identifying compounds that specifically inhibit a metabolic target site or pathway in plants. Enzymes which are specifically affected by the method of the invention include plant pyrimidine biosynthetic pathway enzymes, and particularly the enzymes involved in the conversion of orotate to uridine-5′-monophosphate (UMP). Further, the invention relates to a method for control of undesirable monocotyledenous and dicotyledenous plant species.

### CROSS REFERENCE TO RELATED APPLICATION

This application claims priority to U.S. Provisional Application Ser. No. 60/218,193 filed Jul. 14, 2000 which is herein incorporated by reference in its entirety.

### FIELD OF THE INVENTION

This invention relates to a method for identifying compounds that specifically inhibit a metabolic target site or pathway in plants. Enzymes which are specifically affected by the method of the invention include plant pyrimidine biosynthetic pathway enzymes, and particularly the enzymes involved in the conversion of orotic acid to uridine-5′-monophosphate (UMP). Further, the invention relates to a method for control of undesirable monocotyledenous and dicotyledenous plant species.

### BACKGROUND OF THE INVENTION

Currently, there are only 15 compounds among the commercial herbicides that are known to inhibit a particular protein site within a biochemical pathway (Duke, S. O. (1990) 87:263-271). This factstrongly suggests that many novel herbicidal protein sites are waiting to be discovered. A method of identifying herbicides that target the pyrimidine biosynthesis pathway is described herein. To date, there are no known herbicides that inhibit this pathway.

Methods to determine herbicidal activity typically involve spraying test compounds on whole plants or seeds, and assessing plant injury. Whole plant or in vivo screening requires time, space, and significant quantities of compound. Screening methods that utilize detection of specific enzymes in a pathway provide a useful way of rapidly and efficiently assessing the herbicidal activity of compounds prior to in vivo testing.

Therefore, a method for identifying compounds that specifically inhibit a metabolic target site or pathway in plants is highly desirable. Specifically, there is a need in the art to identify herbicidal compounds that inhibit enzymes in the plant pyrimidine biosynthetic pathway. A method for identifying potential herbicidal compounds that inhibit the enzymes of the plant pyrimidine pathway has now been found and is described herein. Further, it has been found that said compounds are useful for the control of undesirable monocotylenous and dicotyledenous plant species.

### BRIEF SUMMARY OF THE INVENTION

The present invention relates to a method for identifying compounds that inhibit uracil biosynthesis, wherein the method includes the following steps:

i) applying test compounds to plant tissue in the presence of pyrimidine biosynthetic pathway intermediates or end-products;

ii) applying test compounds to plant tissue in the absence of pyrimidine biosynthetic pathway intermediates or end-products;

iii) comparing the effects of the test compound from step i) with the effects of the test compounds from step ii); and

iv) noting the test compounds in step iii) that show reversed effects in the presence of said intermediates or end-products.

In addition, the present invention relates to an herbicidally active compound having the formula I:

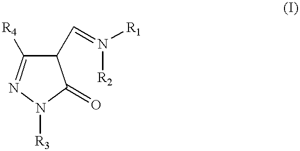

wherein:

R₁ is hydrogen or C₁-C₆-alkyl unsubstituted or substitued with one to three of the following radicals: cyano, nitro, hydroxyl, mercapto, amino, carboxyl, C₁-C₄-alkoxy, C₁-C4-haloalkoxy, C₁-C₄-alkoxycarbonyl or C₁-C₆-alkylthio;

R₂ is hydroxy, OR₅, aryl, arylmethylene, hetaryl, hetarylmethylene unsubstituted or substitued with one to three of the following groups: halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, and C₁-C₆-alkoxycarbonyl;

R₃ is C₁-C₄-alkyl or hydrogen;

R₄ is hydrogen or hydroxy; and

R₅ is C₁-C₆-alkylcarbonyl or a formyl-moiety bonded to the structure via the carbon atom;

with the proviso that if R₁ is H, R₂ can also be HNR₆, wherein R₆ is aryl, arylmethylene, hetaryl, hetarylmethylene unsubstituted or substituted with halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, or C₁-C₆-alkoxycarbonyl;

or an agriculturally acceptable salt or ester thereof.

Moreover, the present invention relates to compositions and methods of using and making the compounds of formula (I).

Furthermore, the present invention relates to a process for production of pesticidal agents comprising the following steps:

a) identification of the uracil biosynthesis inhibitor comprising

i) applying test compounds to plant tissue in the presence of pyrimidine biosynthetic pathway intermediates or end-products;

ii) applying test compounds to plant tissue in the absence of pyrimidine biosynthetic pathway intermediates or end-products;

iii) comparing the effects of the test compound from step i) with the effects of the test compounds from step ii); and

iv) noting the test compounds in step iii) that show reversed effects in the presence of said intermediates or end-products; and

b) preparing a pesticidal composition comprising the pesticidal agent identified in step a).

In yet another embodiment, the present invention relates to a method for identifying the mode of action of a probe compound comprising:

i) treating a plant with one or more radiolabeled pyrimidine biosynthesis intermediates in the presence of probe compound,

ii) treating a plant with one or more radiolabeled pyrimidine biosynthesis intermediates in the absence of probe compound,

iii) determining the percent conversion of said radiolabeled pyrimidine biosynthesis intermediates to radiolabeled pyrimidine biosynthesis products selected from the group consisting of: orotate, uridine-5′-monophosphate, and uracil,

iv) comparing the percent conversion of said intermediates to said products of “ii” with those from step “iii”, and

v) noting specific accumulations of said products in the presence of probe compound as compared to the absence of probe compound.

Advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

### BRIEF DESCRIPTION OF THE FIGURES AND TABLES

FIG. 1. Pyrimidine de novo biosynthesis pathway. This figure depicts a schematic representation of the pyrimidine de novo biosynthetic pathway.

FIG. 2. Pyrimidine salvage pathway. This figure depicts a schematic representation of the pyrimidine salvage pathway, and illustrates how intermediates of uracil biosynthesis are utilized for the biosynthesis of cytidine, thymidine, and RNA.

FIG. 3. Hydroponic studies on corn growth as affected by pyrazolinones. This figure illustrates the activity of several pyrimidine biosynthetic inhibitors, identified by the method of the present invention, and how they affect root and shoot growth when applied to the foliage or the roots of corn plants in a hydroponic system. In addition, the figure illustrates the pyrimidine biosynthetic inhibitors are more xylem mobile than phloem mobile.

FIG. 4. Effect of the compound B on Arabidopsis injury. This figure illustrates schematically the lethal concentration for compound B on Arabidopsis plant growth compared to untreated Arabidopsis plants. The experiment was performed in a microtiter plate format. Each well on the plate contained agar, either in the presence (Row B) or absence (Row A) of decreasing concentrations of the B probe compound from left to right. In this illustration, 5 concentrations of compound B were tested: 500, 100, 50, 16, and 1 μM, as shown in Row B. Row A illustrates that Arabidopsis seeds germinated and grew into healthy plants in the absence of compound B. In the presence of compound B, germination of Arabidopsis seeds was not observed at concentrations of 500, 100, and 50 μM, as shown in Row B. At 16 μM compound B there was partial germination, and at 1 μM compound B there was normal germination. These results illustrate that in the presence of at least 50 uM compound B, Arabidopsis seeds do not germinate, and 50 μM or higher concentrations of compound B are lethal to plant growth.

FIG. 5. Reversal test of the inhibition by probe compound on plant growth with purines and pyrimidines. The figure illustrates schematically that the lethal effect of compound B on Arabidopsis plant growth can be reversed by the addition of uridine. The experiment was performed in a microtiter plate format. The microtiter plate was divided into columns containing various treatments (left to right), and rows containing decreasing concentrations of the compound B (top to bottom). Each well contained agar and Arabidopsis seeds that had or had not germinated. Column 1 represents control untreated wells, and illustrates that Arabidopsis plants germinate and grow in the absence of compound B. Column 2 represents wells that contain decreasing concentrations of compound B with the highest concentration on top and lowest concentration at the bottom of the plate. As illustrated in column 2, high concentrations of compound B (Rows 1-3) are lethal as they inhibit the germination of Arabidopsis, and lower concentrations of compound B (Row 4-5) are not as lethal. Column 3 represents wells that contain the same concentrations of compound B as shown in column 2, with the addition of 100 μM uridine. Column 3 illustrates that in the presence of uridine at intermediate and low concentrations of compound B, Arabidopsis plants germinated and grew in a healthy manner. The results are similar to control untreated plants observed in column 1. Further, column 3 illustrates that at the highest tested concentration of compound B (Row 1, column 3) uridine does not reverse the lethal effects of compound B. However, at lower concentrations (Rows 2-4, column 3), uridine reversed the lethal effects of compound B. Columns 4-7 illustrate the effect that adenine, cytosine, guanine, and thymidine have on the germination and growth of Arabidopsis plants when combined with compound B. As illustrated in columns 4-7, combining adenine, cytosine, guanine, and thymidine with compound B did not reverse the lethal effects of compound B either on Arabidopsis germination or plant growth. This figure illustrates that uridine alone can reverse the lethal effects of the compound B, while other pyrimidines or purines do not reverse the lethal effects of the compound B.

FIG. 6. HPLC profiles of [¹⁴C]-carbamoyl aspartate incorporation in the uracil biosynthesis pathway. The figure illustrates schematically the observed HPLC profiles of radiolabeled carbamoyl aspartate incorporation in the de novo pyrimidine biosynthesis pathway, either in the presence or absence of the compound B. Soybean cell cultures were treated with or without 100 μM compound B plus radiolabeled carbamoyl aspartate, the starting intermediate of the de novo pyrimidine biosynthesis pathway. The radiolabeled signal on the carbamoyl aspartate can easily be followed and detected. The incorporation of radiolabeled carbamoyl aspartate was detected 48 hours after treatment by extracting a crude preparation from treated cell cultures, and analyzing this extract using HPLC. Uridine, uracil, carbamoyl aspartate, UMP, and orotic acid were used as standards to determine the retention time of each intermediate of the de novo pyrimidine biosynthesis pathway. The standard retention times for these intermediates were: 34 min uracil and uridine, 7-9 min carbamoyl aspartate, 11-13 min UMP, and 17-19 min orotic acid. Each point represents the average from two experiments. The top diagram illustrates the HPLC profile for control soybean cells treated only with radiolabeled carbamoyl aspartate and shows two peaks—one major peak at 3-5 min and another peak at 7-9 min. The top diagram illustrates that most of the radiolabeled carbamoyl aspartate is incorporated into uracil/uridine in untreated control soybean cells. The bottom diagram illustrates that in soybean cells treated with 100 μM compound B, the HPLC profile contains 3 peaks—two small peaks at 3-5 min and 11-13 min and one larger peak at 16-19 min. Thus, in contrast to the top diagram, the bottom diagram illustrates that most of the radiolabeled carbamoyl aspartate is incorporated into orotic acid when cells are treated with compound B. The figure illustrates that whereas in untreated control cells most radiolabeled carbamoyl aspartate is incorporated into uracil/uridine, very little is incorporated into uracil/uridine with the addition of compound B. The figure further illustrates that most of the radiolabeled carbamoyl aspartate is incorporated into orotic acid in the presence of compound B. These results illustrate that compound B inhibits the biosynthesis of uracil/uridine, and suggests that compound B inhibits the conversion of orotic acid to uracil/uridine causing orotic acid to accumulate.

FIG. 7. Effect of increasing concentrations of compound B on the incorporation of ¹⁴C-carbamoyl aspartate into uracil and orotate in soybean cell cultures. The figure illustrates graphically the effect of increasing concentrations of compound B on the incorporation of radiolabeled carbamoyl aspartate into uracil/uridine and orotic acid. This figure summarizes the HPLC profiles observed on soybean cell cultures treated with or without compound B plus radiolabeled carbamoyl aspartate. The methods used are the same as those used in the experiments for FIG. 6, which are described elsewhere herein. The percent radiolabeled carbamoyl aspartate incorporated into uracil/uridine and orotic acid were determined for each tested concentration of compound B. The figure on the left illustrates and compares the percent incorporated radiolabeled carbamoyl aspartate into uracil/uridine (y-axis) between control and increasing concentrations of compound B (x-axis). The graph on the left illustrates that in control and at 1 and 10 μM probe compound, there was 80-90% incorporation of radiolabeled carbamoyl aspartate into uracil/uridine. At 50 and 100 μM compound B, the graph on the left illustrates there was only 25 and 10% incorporation of radiolabeled carbamoyl aspartate into uracil/uridine. The graph on the right illustrates that in control and at 1 and 10 μM compound B there was 0-5% incorporation of radiolabeled carbamoyl aspartate into orotic acid, and at 50 and 100 μM compound B there was 45-60% incorporation of radiolabeled carbamoyl aspartate into orotic acid. Combined, the results on the two graphs indicate that at at 50μM and 100 μM compound B, there is less incorporation of radiolabeled carbamoyl aspartate into uracil/uridine, and greater incorporation into orotic acid compared to untreated control cells.

FIG. 8. In vitro enzymatic conversion of ¹⁴C-orotate to UMP over time in control corn root tissue. The figure illustrates the enzymatic conversion of orotate to UMP over time in a cell free enzyme extraction from corn tissue. Plant tissue is ground in liquid nitrogen to a fine powder, and enzyme is extracted with a buffer. The graph illustrates the percent conversion of orotate into UMP (y-axis) over time (x-axis). The graph further illustrates that 15 minutes after treatment, 50% of the radiolabeled orotate is converted to UMP by corn enzyme, and by 30 minutes after treatment 100% of the radiolabeled orotate is converted to UMP. The results on this graph illustrate methods by which OPRTase/ODCase enzyme activity is measured over time.

FIG. 9. Enzymatic conversion of ¹⁴C-orotate to UMP with increasing concentrations of phosphoribosyl pyrophosphate (PRPP) in corn root tissue. The figure illustrates schematically the dependence of the enzymatic activity of OPRTase and ODCase on PRPP. The methods for extracting OPRTase and ODCase activity are described in FIG. 8, and elsewhere herein. The percent conversion of orotate into UMP (y-axis) with increasing concentrations of PRPP (x-axis) 30 minutes after treatment (MAT) in a cell free extract from untreated control corn root tissue are shown. The graph illustrates that at concentrations below 50 μM PRPP, there is 0-40% conversion of orotate to UMP, and at concentrations above 100 μM PRPP there is 80-100% conversion of orotate to UMP 30 MAT. The figure demonstrates that OPRTase/ODCase enzyme activity is dependent on a certain concentration of PRPP that is necessary for orotate to be converted to UMP.

FIG. 10. Comparison between the percent inhibition of in vitro enzymatic conversion of orotate to UMP by the compound B and barbiturate. The figure illustrates schematically the effect of probe compound and barbiturate on the enzymatic activity of OPRTase and ODCase. The methods used to test enzyme activity are described in FIGS. 8, 9, and elsewhere herein. The graph on the left illustrates the percent radiolabeled orotate remaining after a 15 minute incubation with a cell free extract from corn root tissue with 100 μM PRPP. The graph on the left compares the effect of untreated control tissue versus treatment with compound B or barbiturate. The graph on the right illustrates the percent radiolabeled orotate converted to UMP by a cell free extract from corn root tissue with 100 μM PRPP 15 minutes after treatment. As illustrated, compound B does not differ from the untreated control enzyme extract. Treatment of enzyme in control and with compound B resulted in the conversion of 55% orotate into UMP, 15 minutes after treatment. The figure further illustrates that barbiturate, a known inhibitor of OPRTase and ODCase, prevented the conversion of radiolabeled orotate to UMP. The enzyme treated with only barbiturate resulted in a 20% conversion of radiolabeled orotate to UMP 15 minutes after treatment. The results illustrated in the figure indicate that compound B does not affect OPRTase/ODCase activity under these particular assay conditions.

FIG. 11. Comparison between pyrazole aldehyde and barbiturate directed inhibition on the in vitro enzymatic conversion of orotate to UMP. The figure illustrates schematically the effect of the breakdown product of compound B and barbiturate on the enzymatic activity of OPRTase and ODCase. The methods used to test enzyme activity are described in FIG. 10, and elsewhere herein. The graph on the left illustrates the percent inhibition of the conversion of orotate to UMP (y-axis) with increasing concentrations of the breakdown product of probe compound (pyrazole aldehyde) in a cell free extract from corn root tissue. The graph on the right illustrates the percent inhibition of the conversion of orotate to UMP (y-axis) with increasing concentrations of barbiturate, a known inhibitor of OPRTase and ODCase. The figure illustrates that 1 and 2 mM concentrations of the breakdown product of compound B inhibit the conversion of orotic acid to UMP by 20 and 50%. At 1 and 2 mM barbiturate, there is 80 and 100% inhibition of the conversion of orotic acid to UMP. The figure illustrates that the concentration at which there is 50% inhibition of UMP synthesis is 2 mM for the breakdown product of the probe compound, while the concentration at which there is 50% inhibition of UMP synthesis is less than 0.27 mM for barbiturate in the given assay conditions.

FIG. 12. Comparing levels of UMP synthesis over time between the pyrazole aldehyde and control in corn root tissue. The figure illustrates schematically the effect of the breakdown product of compound B on the enzymatic activity of OPRTase and ODCase over time. The methods used to test enzyme activity are described in FIG. 8, and elsewhere herein. The figure illustrates that 100% conversion of radiolabeled orotate to UMP is observed for the untreated control enzyme, whereas only a 50% conversion of orotate to UMP is observed for the breakdown product of compound B. The figure illustrates that the inhibition of the enzymatic activity of OPRTase/ODCase by the breakdown product of compound B is constant over time.

FIG. 13. Effect of several pyrazolinone analogs on the in vitro enzymatic conversion of ¹⁴C-DHOD to orotate and carbamoyl aspartate. The figure illustrates schematically the effect of several probe compounds (compounds B, D, J) on the enzymatic activity of DHO and DHOD. To measure DHO/DHOD activity, corn root tissue was ground to a find powder with liquid nitrogen, and a microsomal preparation containing membrane-bound DHO and DHOD was extracted in a buffered solution. The activity of DHO was measured by the conversion of radiolabeled dihydroorotic acid to carbamoyl aspartate in the extracted microsomal preparation, and the activity of DHOD is measured by the conversion of radiolabeled dihydroorotic acid to orotic acid. The top graph illustrates the effect that several probe compounds had on the conversion of radiolabeled dihydroorotate to orotate compared to control. The bottom graph illustrates the effect of several probe compounds on the conversion of radiolabeled dihydroorotate to carbamoyl aspartate compared to control. The graph illustrates there is no difference between the percent of radiolabeled dihydroorotate converted to carbamoyl aspartate or orotate between the tested probe compounds and the untreated control enzyme extract.

FIG. 14. Stability of the lead uracil biosynthesis inhibitors, compound “B”, compound “C”, compound “G”, and compound “E” in aqueous solution. The figure illustrates the observed half-lives and potential break-down products of the compound “B”, compound “C”, compound “G”, and compound “E” compounds as detected using a combination of mass spectroscopy and NMR analysis. The results indicate that the compound “B” and compound “C” compounds have half-lives of less than 24 hours, while the compound “G” compound had a half-life greater than 24 hours. The compound “E” compound was stable in aqueous solution.

FIG. 15. Proposed structure of the biologically active hydrolysate of compound “B”. The figure illustrates that the purified hydrolysate of compound “B” was found to comprise a complex mixture of isomers as determined by high field NMR analysis. Moreover, the mixture further comprised tautomeric forms of the geometric isomers of the enol form of the pyrazole aldehyde.

FIG. 16. Summary of the possible structures of the breakdown products of compound “B” and its analogs, in addition to their biological activity as uracil biosynthesis inhibitors. The figure illustrates several possible structures of the hydrolysis product of the “B” probe compound detected using a combination of mass spectroscopy and NMR analysis. Several of the structures correspond to parent compounds tested previously herein (e.g., see Table III). Where applicable, detected uracil biosynthesis activity, confirmed using the reversal assay of the present invention, is summarized in the figure.

Table 1.

Greenhouse results for the “B” probe compound. The table illustrates the effect of probe compound on greenhouse grown plants and contains information about the weed spectrum and crop selectivity of compound B at two tested rates (1000 g ai/h and 500 g ai/h). The table demonstrates that compound B affects plant growth when applied pre-emergence or post-emergence.

Table 2.

Reversal of Herbicidal Activity of the “B” probe compound on Arabidopsis. The table illustrates the injury effect of compound B and the reversal effects through the inclusion of several possible antidotes in the growth medium (e.g., uracil, uridine, UMP, AMP, adenine, cytosine, guanine, thymine, and xanthine). In this figure, 50 μM was determined to be the lethal concentration of the “B” probe compound, and the resulting injury was reversed by the addition of uracil, uridine, and UMP, specifically.

Table 3.

Several pyrazolinone analogs and their herbicidal effect. This table illustrates the effect of several probe compounds on greenhouse grown foxtail weed, Arabidopsis injury in a microtiter plate and the observed injury reversal with uracil. In addition, the table also illustrates the injury effect of root applied probe compound on hydroponically grown corn plants. This table illustrates the minimal chemical structural components necessary for probe compound to cause plant injury and reversal by uracil.

Table 4.

Several pyrazolinone analogs and their herbicidal effect. This table illustrates the effect of several probe compounds on greenhouse grown foxtail weed and observed Arabidopsis injury in a microtiter plate and the observed injury reversal with uracil. This table, in conjunction with Table 3, illustrates the minimal chemical structural components necessary for probe compound to cause plant injury and reversal by uracil.

### DETAILED DESCRIPTION OF THE INVENTION

The present invention may be understood more readily by reference to the following detailed description of exemplary embodiments of the invention and the examples included therein.

Before the present compounds, compositions, and methods are disclosed and described, it is to be understood that this invention is not limited to specific synthetic methods of making the compounds or compositions described herein that may of course vary. It is also to be understood that the terminology used herein is for the purpose of describing particular embodiments only and is not intended to be limiting.

In this specification and in the claims that follow, reference will be made to a number of terms that shall be defined to have the following meanings:

DNA=desoxy-ribonucleic acid

OMP=orotidine-5′-monophosphate

PRPP=phosphoribosyl pyrophosphate

RNA=ibonucleic acid

UMP=uridine-5′-monophosphate

UDP=uridine-5′-diphosphate

UTP=uridine-5′-triphosphate

The term “pyrimidine biosynthesis” means the conversion of carbamoyl phosphate and aspartic acid to form UMP, UDP, UTP, CTP, and thymidine.

The term “UMP biosynthesis” means the conversion of carbamoyl phosphate and aspartic acid to form UMP.

A “probe compound” is a compound used in the methods described herein which potentially inhibits either directly or indirectly one or more of the plant pyrimidine biosynthetic pathway enzymes.

The term “antidote compound” may be defined generally to include any compound capable of reversing the uracil biosynthesis inhibitory effects of the probe compounds of the present invention. Such antidote compounds may include the following, non-limiting compounds: uracil, uridine, UMP, orotate, N-carbamoylaspartate, NAD+, PRPP, L-dihydroorotate, asparate, carbamoyl phosphate, OMP, UDP, and UTP, for example.

The terms “herbicide” and “herbicidal” are used herein to denote the inhibitive control or modification of undesired plant growth. Inhibitive control and modification include all deviations from natural development, such as, total killing, growth retardation, defoliation, desiccation, regulation, stunting, tillering, stimulation, leaf bum and dwarfing.

The term “herbicidally effective amount” is used to denote any amount which achieves such control or modification when applied to the undesired plants themselves or to the area in which these plants are growing.

The term “plants” is intended to include seeds, seedlings, germinated seeds, emerging seedlings, plant tissue (e.g., meristematic tissue, root tissue, stem tissue, flower tissue, cotyledon tissue, shoot tissue, callus, etc,), plant cultures, plant cells, established vegetation, including both roots and above-ground portions, and preferably Arabidopsis plants and seeds.

The term “agriculturally acceptable salt” is easily determined by one of ordinary skill in the art and includes hydrohalide, acetic, sulfonic, phosphonic, inorganic and organic acid salts. Moreover, “salt” as used herein includes salts that can form with, for example, amines, alkali metal bases and alkaline earth metal bases or quaternary ammonium bases, including zwitterions. Suitable alkali metal and alkaline earth metal hydroxides as salt formers include the hydroxides of lithium, sodium, potassium, magnesium or calcium. Illustrative examples of amines suitable for forming ammonium cations are ammonia as well as primary, secondary and tertiary amines such as methylamine, ethylamine, n-propylamine, isopropylamine, the four isomeric butylamines, n-amylamine, isoamylamine, hexylamine, heptylamine, octylamine, nonylamine, decylamine, pentadecylamine, hexadecylamine, heptadecylamine, octadecylamine, methyl ethylamine, methyl isopropylamine, methyl hexylamine, methyl nonylamine, methyl pentadecylamine, methyl octadecylamine, ethyl butylamine, ethyl heptylamine, ethyl octylamine, hexyl heptylamine, hexyl octylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines such as pyridine, quinoline, isoquinoline, morpholine, piperidine, pyrrolidine, indoline, quinuclidine and azepine; primary arylamines such as anilines, methoxyanilines, ethoxyanilines, o-, m- and p-toluidines, phenylenediamines, benzidines, naphthylamines and o-, m- and p-chloroanilines.

In general, “herbicidally effective amount” means the amount of herbicide needed to achieve an observable herbicidal effect on plant growth, including the effects of plant necrosis, plant death, growth inhibition, reproduction inhibition, inhibition of proliferation, and removal, destruction, or otherwise diminishing the occurrence and activity of a plant. Typically, the herbicide is useful in controlling monocotyledonous and dicotyledonous weeds. One of ordinary skill in the art will recognize that the potency and, therefore, an “herbicidally effective amount,” can vary for the various herbicidal compounds/compositions used in the invention. “Non-herbicidally effective amount” shall mean that amount of herbicide that is not an herbicidally effective amount.

“Area intended for crop plants” shall mean the area of soil and/or water in which crop plants are already growing, the area in which the seed of those crop plants already has been sown, and the area intended for growing crop plants before the seed is sown.

As used throughout, the term “contacting” is used to mean that the undesired plant and/or the area intended for crop plants (i.e. soil, water) has contact with the present compound(s) or composition(s) by application methods known in the art. As such, “contacting” includes both direct contact (applying the composition directly on the plant) and indirect contact (applying the composition to the area intended for crop plants whereupon the plant incorporates the active ingredients). This contact can take place before the undesired plant emerges from the soil (“pre-emergence” or “PRE”) or after the undesired plant emerges from the soil (“post-emergence” or “POST”). Additionally, all compounds of the present invention need not contact the undesired plant simultaneously as the present invention is intended to include application of the desired components sequentially, as long as a synergistic effect is achieved.

The term “crop plant” as used herein includes terrestrial and aquatic crop plants. Exemplary crop plants include cereal crops (wheat, rye, barley, oats), plantation crops, (rubber, pineapple, coffee, bananas, tea), orchard crop plants (citrus fruit trees, apple trees, peach trees, pear trees, nut trees, gum trees, coconut trees, olive trees), and other crop plants including, but not limited to, rice, corn, sorghum, radish, chinese cabbage, cotton, sugar cane and soybeans.

By “undesired plant” is meant to include seeds, tubers, rhizomes, and foliage broad-leaf and gramineous plants, including monocotyledon weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus and Apera; monocotyledon cultures of the genera: Oryza, Zea, Triticum, Hordeum, Avena, Secale, Sorghum, Panicum, Saccharum, Ananas, Asparagus and Allium; dicotyledon weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver Centaurea, Trifolium, Ranunculus and Taraxacum; and dicotyledon cultures of the genera: Gossypium, Glycine, Beta, Daucus, Phaseolus, Pisum, Solanum, Linum, Ipomoea, Vicia, Nicotiana, Lycopersicon, Arachis, Brassica, Lactuca, Cucumis and Cucurbita. However, the use of the active compounds/compositions according to the invention is in no way restricted to these genera, but also extends in the same manner to other plants.

The “compounds” referenced herein refer to compounds tested and/or identified using the method of the present invention. Where applicable, the chemical structure of each compound is shown that are mentioned in the further application as compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, compound “Z”(for chemical structures see e.g. Table 3 and 4). In addition, the following table may be referenced for each compounds chemical name.

| Compound | Chemical Name |
|---|---|
| A | 4-[[(2,4-dichlorophenyl)amino]methylene]-2,4- |
| | dihydro-3H-pyrazol-3-one |
| B | 4-[[(2,4-difluorophenyl)amino]methylene]-2,4- |
| | dihydro-3H-pyrazol-3-one |
| C | 4-[[2-cyanophenyl)amino]methylene]-2,4-dihydro- |
| | 3,4-pyrazol-3-one |
| D | 2,3-dihydro-3-oxo-1H-pyrazole-4-carboxaldehyde-4- |
| | oxime |
| E | 4-[(dimethylamino)methylene]-2,4-dihydro-3H- |
| | pyrazol-3-one |
| F | Hydrochloride of compound “E” |
| G | 4-[[(2,4-difluorophenyl)amino]methylene]-2,4- |
| | dihydro-2-methyl-3H-pyrazol-3-one |
| H | Hydrochloride of |
| | 4-[(dimethylamino)methylene]-2,4-dihydro-2- |
| | methyl-3H-pyrazol-3-one |
| I | 2-aminobenzonitrile |
| J | 4,5-dihydro-5-oxo-1H-pyrazole-4-carboxylic acid; or |
| | 2,3-dihydro-3-oxo-1H-pyrazole-4-carboxylic acid |
| K | 4-[[[2-(trifluoromethyl)phenyl]amino] |
| | methylene]-2,4-dihydro-3H-pyrazol-3-one |
| L | 4-[[[3-(trifluoromethyl)phenyl]amino] |
| | methylene]-2,4-dihydro-3H-pyrazol-3-one |
| M | 4-[[(4-methoxyphenyl)amino]methylene]-2,4- |
| | dihydro-3H-pyrazol-3-one |
| N | 4-[[(3-chlorophenyl)amino]methylene]-2,4-dihydro- |
| | 3H-pyrazol-3-one |
| O | 4-[[(1,5-dihydro-5-oxo-4H-pyrazol-4- |
| | ylidene)methyl]amino]benzoic acid, methyl ester |
| P | 4-[[(2-methoxyphenyl)amino]methylene]-2,4- |
| | dihydro-3H-pyrazol-3-one |
| Q | 4-[[(3-methoxyphenyl)amino]methylene]-2,4- |
| | dihydro-3H-pyrazol-3-one |
| R | 4-[[(4-nitrophenyl)amino]methylene]-2,4-dihydro- |
| | 3H-pyrazol-3-one |
| S | 4-[[(3,5-dichlorophenyl)amino]methylene]-2,4- |
| | dihydro-3H-pyrazol-3-one |
| T | 4-[[(1,5-dihydro-5-oxo-4H-pyrazol-4- |
| | ylidene)methyl]amino]-N,N- |
| | dimethylbenzenesulfonamide |
| U | 4-[[[4-(trifluoromethoxy)phenyl]amino] |
| | methylene]-2,4-dihydro-3H-pyrazol-3-one |
| V | 3-[[(1,5-dihydro-5-oxo-4H-pyrazol-4- |
| | ylidene)methyl]amino]-2-thiophenecarboxylic acid, |
| | methyl ester |
| W | 4-[[(3-pyridylmethyl)amino]methylene]-2,4-dihydro- |
| | 3H-pyrazol-3-one |
| X | 4-[[(2,4-difluorophenyl)methylamino] |
| | methylene]-2,4-dihydro-3H-pyrazol-3-one |
| Y | 5-hydroxy-1H-pyrazole-4-carboxaldehyde, 2,4- |
| | difluorophenylhydrazone |
| Z | 4-[[(2,4-difluorophenyl)amino]methylene]-3,5- |
| | pyrazolidinedione |

The pyrimidine biosynthetic pathway is a biochemical process by which all living organisms make pyrimidines. Uridine, cytidine, and thymidine are all examples of pyrimidines. The pyrimidines are essential components of all organisms including plants. Pyrimidines are the building blocks of RNA and DNA, which encode genetic information. Inhibition of the pyrimidine biosynthetic pathway in any organism results in inhibition of RNA and DNA synthesis.

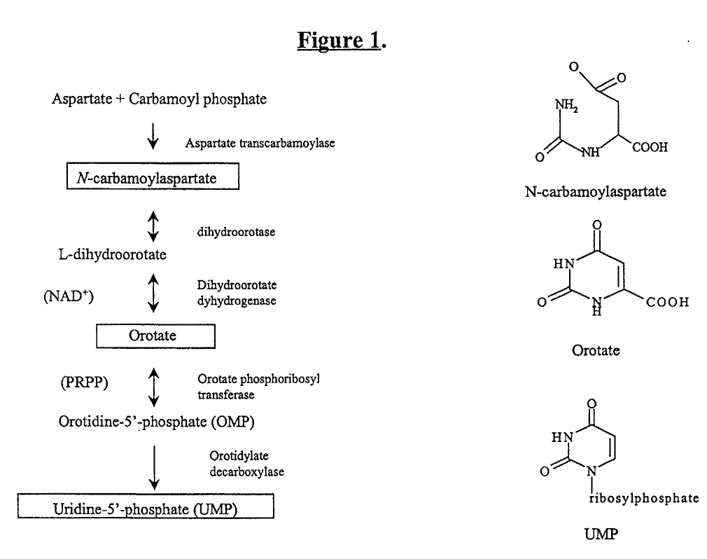

The pyrimidine pathway begins with the synthesis of UMP. UMP is then further phosphorylated to yield UDP and UTP, which subsequently serve as the starting point for thymidine biosynthesis or for CTP biosynthesis. Thede novo pathway for UMP biosynthesis is shown in FIG. 1. and starts with aspartate and carbamoyl phosphate reacting to form N-carbamoyl aspartate. Subsequently, water is removed, the ring closes and dihydroorotate is formed. Dihydroorotate is oxidized to yield orotate. A ribose-5-phosphate side chain, provided by phosphoribosyl-pyrophosphate (PRPP), is then attached to orotate to form orotidine monophosphate (OMP). OMP is decarboxylated to yield uridine-5′-monophosphate (UMP).

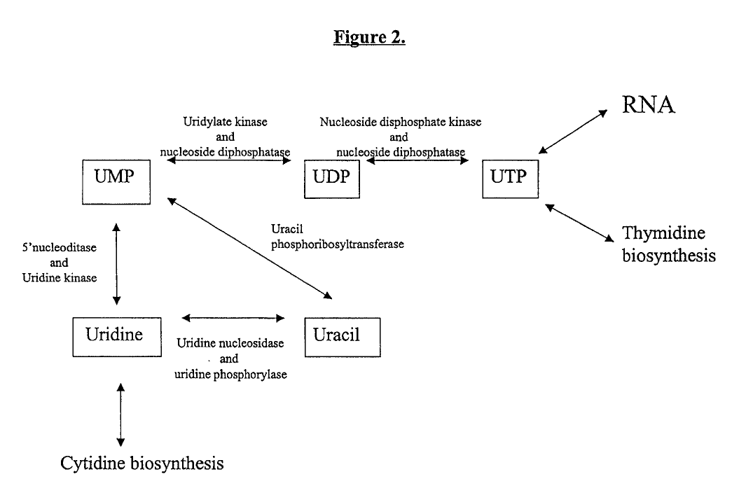

UMP is then converted to UDP, UTP, uracil, and uridine, via the pyrimidine salvage pathway involving various kinases and dephosphatases that either phosphorylate or dephosphorylate UMP and its derivatives. The salvage pathway (FIG. 2) serves as a recycling pathway between UMP, UDP, UTP, uracil and uridine; whereas the de novo pathway serves to make UMP from its starting materials, carbamoyl phosphate and aspartic acid.

In plants, all enzymes involved in the de novo and salvage biosynthesis of UMP and uracil are located in the chloroplast with the exception of dihydroorotate dehydrogenase (DHOD) (Doremus, H. D. (1986). Arch. Of Biochem. and Biophysics 250(1):112-119; Doremus, et al. (1987) Plant Physiol. 83:657-658, Doremus, H. D. et al. (1995) Plant Physiol. 79:856-861.). DHOD, the enzyme that oxidizes dihydroorotate to orotate, is located in the outer membrane of the mitochondria, which is common among all organisms.

There are also genetic differences in the organization of the UMP biosynthesis pathway enzymes between organisms. In higher eukaryotes such as in Arabidopsis thaliana, Drosophila melanogaster, and humans, the last two enzymes that convert orotate to UMP, orotate phosphoribosyl transferase and decarboxylase (OPRTase/ODCase), exist as a large bifunctional enzyme, commonly referred to as UMP synthase, encoded by one gene (Nasr, F. et al. (1994) Mol. Gen. Genet. 244:23-32). In microorganisms such as Escherichia coli, Bacillus subtilis, and yeast, these enzymes exist as separate proteins and are encoded by more than one gene.

The pyrimidine pathway has been studied in great detail in animals and certain microorganisms. There are several known inhibitors that target the OPRTase/ODCase enzymes in the de novo biosynthetic pathway of UMP in animals. Conversely, these inhibitors were discovered as anticancer drugs and antimicrobial agents (Niedzwicki, J. G. et al. (1984) Biochem. Pharm. 33:2383-2395).

Barbiturate is a known inhibitor of dihyroorotate dehydrogenase (DHOD, Kₘ 402 uM), OPRTase (Kₘ 1.5 uM), and ODCase, (Kₘ 16 uM) in rat brain tissue with Kᵢ values of 412 uM, 18 uM, and 75 uM, respectively. The ribotide of barbiturate is a potent inhibitor of ODCase activity, with a Kᵢ of 4.1 nM in rat brain tissue, suggesting that ribophosphorylation increases inhibition potency of this compound (Potvin, B. W. et al.(1978) Biochem. Pharm. 27:655-665). In addition, barbiturate inhibits uridine phosphorylase (Kₘ 420 uM) activity in the salvage pathway with a Kᵢ of 786 uM. Barbiturate is a competitive inhibitor of all four enzymes. 5-Phenylbarbituric acid and its derivatives are another group of potent and specific inhibitors of uridine phosphorylase with Kᵢ values ranging around 2 nM (Naguib, F. N. et al. (1993) Biochem. Pharm. 46(7):1273-1283).

2-Thiouracil and 2-thiouridine are also examples of uracil biosynthesis inhibitors. 2-Thiouracil is a potent competitive inhibitor of rat liver uridine phosphorylase. 2-Thiouridine is a potent inhibitor of uridine kinase in the salvage pathway. In addition, 2-thiouracil is a good substrate for OPRTase in bacteria, and is incorporated into RNA. Thiouracil is not an inhibitor of ODCase, but 2-thio-UMP is an inhibitor of ODCase activity. (Lindsay, R. H. et al. (1974) Biochem. Pharm. 23:2273-2281). Again, ribophosphorylation of substrate/inhibitor seems to be critical for ODCase activity and inhibition.

6-Azauridine is a potent inhibitor of pyrimidine biosynthesis (Saenger, W. et al. (1973) Nature 242:610-612). When converted to 6-azauridine-5′-monophophate (6-AMP) by uridine kinase, it inhibits ODCase, and stops the conversion of orotate to UMP.

Compounds that inhibit other protein targets within the pyrimidine biosynthetic pathway are also known. For example, AAL toxin and N-(phosphononacetyl)-L-aspartate are known inihibitors of aspartate carbamoyltransferase (see Gilchrist, D. G., “Molecular mode of action.”, in Toxins and Plant Pathogensis, J. M. Daly and B. J. Deverell, eds., New York:Academic Press, pp.81-136, (1983); and M. A. Acaster, et al., J. Exper. Botany, 40:1121-1125, (1989), respectively). In addition, the compound 8-chloro-phenoxyquinoline LY214352 is a known inhibitor of dihydroorotate dehydrogenase and is described as a fungicide (see G. Gustafson, et al., in “Modern Fungicides and Antifungal Compounds II”, Chapter 11, Intercept Limited, Andover, UK, pp.93-100, (1999)).

Some of the inhibitors that target the UMP biosynthesis pathway in animals also inhibit UMP biosynthesis at the enzyme level in plants. For example, barbiturate inhibits plant OPRTase/ODCase activity as is described in the examples below. However, barbiturate is a poor herbicide since it does not cause whole plant injury. Surprisingly, a method to identify compounds that are both herbicidal and target the UMP biosynthesis pathway has now been found.

The present invention provides a method for identifying compounds capable of inhibiting pyrimidine biosynthesis, either directly or indirectly. The method of the present invention may be used for identifying compounds capable of inhibiting the pyrimidine de novo biosynthesis pathway, either directly or indirectly. Furthermore, the method of the present invention may be used for identifying compounds capable of inhibiting the pyrimidine salvage biosynthesis pathway, either directly or indirectly. Specifically, the inventive method is useful for identifying compounds capable of inhibiting, either directly or indirectly, cytidine, uridine, uracil, and/or thymidine biosynthesis, in addition to, the biosynthesis of known pyrimidine analogs and/or variants, though preferably inhibitors of uracil biosynthesis.

The present inventive method may be used for the identification of compounds that inhibit pyrimidine biosynthesis through, either direct or indirect, inhibition of aspartate transcarbamoylase, dihydroorotase, dihydroorotate dehydrogenase, orotate phosphoribosyl transferase, orotidylate decarboxylase, and/or UMP synthase, though preferably UMP synthase. Moreover, inventive method may be used for the identification of compounds that inhibit pyrimidine biosynthesis through, either direct or indirect inhibition of 5′nucleosidase, uridine kinase, uridylate kinase, nucleoside diphosphatase, nucleoside diphosphate kinase, nucleoside diphosphatase, uracil phosphoribosyltrasferase, uridine nucleosidase, and/or uridine phosphorylase. It is further contemplated that the identification of compounds that inhibit pyrimidine biosynthesis through, either direct or indirect inhibition of aspartate, carbamoyl phosphate, N-carbamoylaspartate, L-dihydroorotate, NAD+, PRPP, orotate, orotidine-5′-phosphate, and/or uridine-5′-phosphate may be obtained by the method of the invention.

In one embodiment, the present inventive method that may be used for the identification of compounds that inhibit pyrimidine biosynthesis comprises the following steps: i) applying test compounds to plant tissue in the presence of pyrimidine biosynthetic pathway intermediates and/or end-products; ii) applying test compounds to plant tissue in the absence of pyrimidine biosynthetic pathway intermediates and/or end-products; iii) comparing the effects of the test compound from step i with the effects of the test compounds from step ii; and iv) noting the test compounds in step “iii” that show reversed effects in the presence of said intermediates and/or end-products.

The present invention therefore comprises a method of identifying potential inhibitors of the plant pyrimidine biosynthetic pathway.

The plant tissue for this method may be a member selected for the group consisting of: seeds, seedlings, germinated seeds, emerging seedlings plant tissue, meristematic tissue, root tissue, stem tissue, flower tissue, cotyledon tissue, shoot tissue, callus, plant cultures, plant cells, plant vegetation, plant roots, Arabidopsis plants, and Arabidopsis seeds. Other plant tissues are known in the art and are encompassed by the present invention.

The pyrimidine biosynthetic pathway intermediates and/or end-products are preferably selected from the group consisting of: uracil, UMP, uridine, orotate, OMP, aspartate, carbamoyl phosphate, N-carbamoylaspartate, L-dihydroorotate, NAD+, PRPP, UDP, UTP, cytidine, and thymidine. The following pyrimidine biosynthetic pathway intermediates and/or end-products are most preferred, uracil, UMP, and uridine. In this context, the intermediates and/or end-products may be referred to as “antidotes”, based upon their ability to reverse the pyrimidine biosynthesis inhibitory affects of a test compound.

The method of identifying potential inhibitors of the plant pyrimidine biosynthetic pathway may additionally comprise the step of determining the lethal concentration of the identified compound. Moreover, the method of identifying potential inhibitors of the plant pyrimidine biosynthetic pathway may additionally comprise the step of determining the reversal conditions of the identified compounds.

The inhibitor identified by the method may be a phosphorylated compound, or a non-phosphorylated compound. The compound may be metabolically, enzymatically, and/or synthetically phosphorylated using methods known in the art. Alternatively, the inhibitor identified by the method may require further modification prior to becoming an active inhibitor of a plant pyrimidine biosynthetic pathway. Such modifications include, but are not limited to phosphorylation, reduction, oxidation, etc.

Preferably, the inhibitor identified by the method inhibits, either directly or indirectly, pyrimidine biosynthesis. More preferably, the inhibitor identified by the method inhibits, either directly or indirectly, the pyrimidine de novo biosynthesis pathway. Most preferred, are inhibitors identified by the method that inhibit the uracil biosynthesis pathway. Alternatively, the inhibitor identified by the method may inhibit, either directly or indirectly, the pyrimidine salvage biosynthesis pathway.

The inhibitors identified by the method of the present invention may inhibit pyrimidine biosynthesis by either directly or indirectly inhibiting the activity of aspartate transcarbamoylase, dihydroorotase, dihydroorotate dehydrogenase, orotate phosphoribosyl transferase, orotidylate decarboxylase, and/or UMP synthase, though preferably UMP synthase. The inhibitors identified by the method of the present invention may inhibit pyrimidine biosynthesis through, either direct or indirect inhibition of 5′nucleosidase, uridine kinase, uridylate kinase, nucleoside diphosphatase, nucleoside diphosphate kinase, nucleoside diphosphatase, uracil phosphoribosyltrasferase, uridine nucleosidase, and/or uridine phosphorylase. In addition, inhibitors identified by the method of the present invention may inhibit pyrimidine biosynthesis through, either direct or indirect inhibition of aspartate, carbamoyl phosphate, N-carbamoylaspartate, L-dihydroorotate, NAD+, PRPP, orotate, orotidine-5′-phosphate, and/or uridine-5′-phosphate.

The inhibitors identified by the method of the present invention preferably have biological activity. Specifically, the inhibitors identified by the method of the present invention are preferably herbicidal to one or more plant species. Moreover, the invention encompasses inhibitors identified by the method of the present invention that may have fungicide, nematicide, insecticide, and/or anti-bacterial activity.

As mentioned elsewhere herein, organism like insects, share many of the same purine and pyrimidine biosynthetic enzymes as plants, too. As a result, it is expected that compounds identified by the method of the present invention are capable of serving as an insecticide, either directly or indirectly.

Once a compound is identified as an uracil biosynthesis inhibitor, a variety of techniques can be applied to determine whether the compound has insecticidal activity for one or more arthropod species and/or host plants. Specifically, the plants tested may be any plant known in the art, though preferrably, the plants referenced elsewhere herein, and more preferably, the Delta Pine variety of cotton, California Wonder variety of pepper, the Mixed Jewel variety of nasturtium, and the Henderson variety of lima bean. At least one, two, three, four, ten, twenty-five, forty, or one hundred arthropods are applied to each plant evaluated. The arthropods may be applied either directly or indirectly (i.e., via infested leaf sections), and may be selected from the group consisting of Aphis gossypii, Myzus persicae, Aphis fabae, Tetranychus urticae, and Spodoptera eridania. If the arthropods are applied indirectly, the infected leaf sections are removed after 24 hours, post application. Other arthropods are known in the art and may be applicable to the above evaluation.

Once infested, the foliage of the intact plants are dipped into a liquid formulation comprising one or more compounds of the present invention, and optionally amended with 100 ppm Kinetic□ (surfactant). Alternatively, the plant foliage may be dipped in a liquid formulation comprising one or more compounds of the present invention, optionally amended with 100 ppm Kinetic□ (surfactant) prior to infestation. The precise composition of the dipping formulation may be empirically determined based upon the unique stability requirements of the compounds of the present invention. For example, the compound of the present invention may require addition of known stabilizing agents, dispersing agents, surfactants, etc. The mortality of the arthropods on thetreated plants may be determined after at least one, two, three, four, five, or more days.

Once a compound of the present invention is determined to confer resistance to arthropod infestation, the positive compound could then be subjected to secondary evaluations to identify its effective concentration. For example, the plants of the secondary evaluations could be dipped into a liquid formulation comprising one or more compounds of the invention, and optionally amended with 100 ppm Kinetic□ (surfactant), prior, or subsequent to, infestation equal to 1×, ⅓×, and/or 1/30 the rate originally tested in the primary screen, for example.

The skilled artisan would appreciate that the above example is exemplary, and should not be construed as limiting. Moreover, the skilled artisan would appreciate that other methods are known in the art that may be more applicable, or preferred, in certain circumstances. Such methods could readily be substituted for the methods outlined above.

As mentioned elsewhere herein, organism like nematodes, share many of the same purine and pyrimidine biosynthetic enzymes as plants, too. As a result, it is expected that at least some of the compounds identified by the method of the present invention may be capable of serving as a nematicide, either directly or indirectly.

Once a compound is identified as a uracil biosynthesis inhibitor, a variety of techniques can be applied to determine whether the compound has nematicide activity for one or more nematode species and/or host plants. Specifically, the plants tested may be any plant known in the art, though preferrably, the plants referenced elsewhere herein in, and more preferably, a tomato transplant. In one example, the experimental compound is solubilized in a liquid formulation to the required test concentration (ppm as wt AI:wt soil). The resulting test solution containing the compound of the present invention, is drenched into sandy loam soil in a pot with a 3-week-old tomato transplant. One day after treatment with the test solution, the treated soil pots are infested with at least one root-knot nematode Meloidogyne incognita J2 larvae. The nematode larvae may be applied either directly or indirectly (i.e., via infested leaf sections). If the nematode larvae are applied indirectly, the infected leaf sections are removed after 24 hours, post application. The pots are kept in the greenhouse, and 4 weeks following inoculation of nematodes, plant roots are washed free of soil and examined for the presence of galls per root-mass. Other nematodes are known in the art and may be applicable to the above evaluation. The invention encompasses the application of one or more compounds of the present invention to the liquid formulation and/or soil above.

The precise composition of the liquid formulation may be empirically determined based upon the unique stability requirements of the compounds of the present invention. For example, the compound of the present invention may require addition of known stabilizing agents, dispersing agents, surfactants, etc.

Once a compound of the present invention has been shown to inhibit nematode infestation, the positive compound of the present invention could then be subjected to secondary evaluations to identify the effective concentration. For example, the test soils of the secondary evaluations could be prepared with a liquid formulation comprising one or more compounds of the present invention, at a rate of application ((ppm as wt AI:wt soil) equal to 1×, ⅓×, and/or 1/30 the rate originally tested in the primary screen, for example.

The skilled artisan would appreciate that the above example is exemplary, and should not be construed as limiting. Moreover, the skilled artisan would appreciate that other methods are known in the art that may be more applicable, or preferred, in certain circumstances. Such methods could readily be substituted for the methods outlined above.

As mentioned elsewhere herein, organism like fungi, share also many of the same purine and pyrimidine biosynthetic enzymes as plants. As a result, it is expected that at least some of the compounds identified by the method of the present invention may be capable of serving as a fungicide, either directly or indirectly.

Once a compound is identified as a uracil biosynthesis inhibitor, a variety of techniques can be applied to determine whether the compound has fungicidal activity for one or more fungal species and/or host plants. Specifically, the plants tested may be any plant known in the art, though preferably, the plants referenced elsewhere herein. In one example, the experimental compound of the present invention is solubilized in a liquid formulation to the required test concentration (ppm as wt AI:wt soil). The formulation may or may not contain a surfactant. The resulting test solution containing the formulated (if required) compound of the present invention, is applied exogenously to the plant, and/or soil, and allowed to dry. Later the same day, the treated plant foliage, and/or roots, are inoculated with an appropriate plant fungal pathogen. The plant fungal pathogen may be one, or more, fungal organisms known in the art. The inoculated plants are kept under conditions conducive to infection for 1-5 days, and then are subjected to conditions conducive to post-infection disease development for an additional 1 to 35 days depending upon the unique requirements of the fungal pathogen (if any). Once disease signs/symptoms are observed, the polypeptides of the present invention are evaluated for an observable reduction in disease level relative to an untreated, inoculated control. The skilled artisan would appreciate that the specific innoculation conditions may vary from one organism to another and may need to be empirically determined. The invention encompasses the application of one or more compounds of the present invention to the liquid formulation and/or soil above.

The precise composition of the liquid formulation may be empirically determined based upon the unique stability requirements of the compounds of the present invention. For example, the compound of the present invention may require addition of known stabilizing agents, dispersing agents, surfactants, etc.

The compositions may contain the herbicidally active components mixture in a range from 0.1 parts to 100 parts by weight and may also contain at least one agriculturally acceptable carrier. The carrier may be any natural or synthetic organic or inorganic ingredient that facilitates dispersion of the composition and contact with the plant.

Exemplary carriers include water, aqueous solutions, N-methylpyrrolidone, alcohols (e.g. methanol, ethanol, n-propanol, isopropanol, ethylene glycol, etc.), ketones (e.g. acetone, methyl ethyl ketone, etc.), ethers (e.g. dioxane, tetrahydrofuran, ethylene glycol monomethyl ether, diethylene glycol monomethyl ether, propylene glycol monomethyl ether, etc.), aliphatic hydrocarbons (e.g. kerosene, lamp oil, fuel oil, machine oil, etc.), aromatic hydrocarbons (e.g. benzene, toluene, xylene, solvent naphta, methylnaphthalene, etc.), halogenated hydrocarbons (e.g. dichloromethane, chloroform, carbon tetrachloride, etc.), acid amides (e.g. dimethylformamide, dimethylacetamide, etc.), esters (e.g. ethyl acetate, butyl acetate, fatty acid glycerol ester, etc.), nitrites (e.g. acetonitrile, propionitrile, etc.), and combinations thereof.

The compositions of the present invention may also contain one or more surfactants to increase the biological effectiveness of the active ingredient. Suitable surface active ingredients include surfactants, emulsifying agents, and wetting agents. A wide range of surfactants is available and can be selected readily by those skilled in the art from “The Handbook of Industrial Surfactants,” 2nd Edition, Gower (1997), which is incorporated herein by reference in its entirety for all purposes. There is no restriction on the type or chemical class of surfactant that can be used. Nonionic, anionic, cationic and amphoteric types, or combinations of more than one of these types, are all useful in particular situations.

Among nonionic surfactants, exemplary classes include polyoxyethylene alkyl, alkyne, alkynyl or alkylaryl ethers, such as polyoxyethylene primary or secondary alcohols, alkylphenols or acetylenic diols; polyoxyethylene alkyl or alkyne esters, such as ethoxylated fatty acids; sorbitan alkylesters, whether ethoxylated or not; glyceryl alkylesters; sucrose esters; and alkyl polyglycosides. Exemplary anionic surfactant classes include fatty acids, sulfates, sulfonates, and phosphate mono- and diesters of alcohols, alkylphenols, polyoxyethylene alcohols and polyoxyethylene alkylphenols, and carboxylates of polyoxyethylene alcohols and polyoxyethylene alkylphenols. These can be used in their acid form but are more typically used as salts, for example sodium, potassium or ammonium salts.

Cationic surfactants classes include polyoxyethylene tertiary alkylamines or alkenylamines, such as ethoxylated fatty amines, quaternary ammonium surfactants and polyoxyethylene alkyletheramines. Representative specific examples of such cationic surfactants include polyoxyethylene (5) cocoamine, polyoxyethylene (15) tallowamine, distearyldimethylammonium chloride, N-dodecylpyridine chloride and polyoxypropylene (8) ethoxytrimethylammonium chloride. Many cationic quaternary ammonium surfactants of diverse structures are known in the art to be useful in combination with herbicides and can be used in compositions contemplated herein.

Suitable emulsifying agents and wetting agents include, but are not limited to, ionic and nonionic types such as polyacrylic acid salts, lignosulphonic acid salts, phenolsulphonic or naphthalenesulphonic acids, products of polycondensation of ethylene oxide with fatty alcohols, fatty acids or fatty amines, substituted phenols (especially alkylphenols or arylphenols), sulphonosuccinic acid ester salts, taurine derivatives (especially alkyl taurates), phosphoric esters of alcohols or products of polycondensation of ethylene oxide with phenols, esters of fatty acids with polyhydric alcohols, and derivatives having sulphate, sulphonate and phosphate groups, of the compounds above.

The compositions of the present invention are suitable for all methods of application used in agriculture, including preemergence application, post-emergence application, and seed dressing. Thus it is to be understood that the present invention includes the application of the individual herbicidal constituents of the compositions of the present invention simultaneously in the same application or sequentially, as in separate applications, to the undesired plant or area intended for crop plants. Such sequential applications may be performed by applying the combination of herbicides individually within a one day period or less, such as separate applications of the individual herbicides within less than 1 hour, less than 5 hours, less than 10 hours, less than 14 hours, or less than 17 hours.

Once a compound of the present invention is determined to confer resistance to fungal infestation, the positive compound of the present invention could then be subjected to secondary evaluations to identify the effective concentration. For example, the test compounds of the secondary evaluations could be prepared with a liquid formulation comprising one or more compounds of the present invention, at a rate of application ((ppm as wt AI:wt soil) equal to 1×, ⅓×, and/or 1/30 the rate originally tested in the primary screen, for example.

The skilled artisan would appreciate that the above example is exemplary, and should not be construed as limiting. Moreover, the skilled artisan would appreciate that other methods are known in the art that may be more applicable, or preferred, in certain circumstances. Such methods could readily be substituted for the methods outlined above.

The present invention furthermore encompasses pesticidal compositions comprising at least an uracil biosynthesis inhibitor identified by the methods mentioned above.

Another embodiment of the present invention relates to a method of identifying the mode of action of the identified inhibitor of the present invention.

In a preferred embodiment this method may comprise the following steps:

i. Treating a plant with one or more radiolabeled pyrimidine biosynthesis intermediates in the presence of probe compound,

ii. Treating a plant with one or more radiolabeled pyrimidine biosynthesis intermediates in the absence of probe compound,

iii. Determining the percent conversion of said radiolabeled pyrimidine biosynthesis intermediates to radiolabeled pyrimidine biosynthesis products selected from the group consisting of: orotate, UMP, and uracil,

iv. Comparing the percent conversion of said intermediates to said products of “ii” with those from step “iii”, and

v. Noting specific accumulations of said products in the presence of probe compound as compared to the absence of probe compound.

The plant tissue for this mode of action method may be a member selected from the group consisting of: seeds, seedlings, germinated seeds, emerging seedlings, plant tissue, meristematic tissue, root tissue, stem tissue, flower tissue, cotyledon tissue, shoot tissue, callus, plant cultures, plant cells, plant vegetation, plant roots, Arabidopsis plants, and Arabidopsis seeds. Other plant tissues are known in the art and are encompassed by the present invention.

The radiolabeled pyrimidine biosynthesis intermediates may be selected from the group consisting of [14C] carbamoyl aspartate, [14C] dihydroorotate, [14C] orotate, [14C]-orotidine-5′-monophosphate, [14C]-uridine-5′-monophosphate, [14C]-uridine-5′-diphosphate, and/or [14C]-uridine-5′-triphosphate and can be purchased (e.g. from MOravek, Biochemicals, Brea, Calif., USA or NEN Life).

The present invention encompasses uracil biosynthesis inhibitors demonstrate herbicidal, fungicidal, insecticidal, nematicidal or acaricidal activity identified using the methods mentioned above.

Uracil biosynthesis inhibitors are useful, for example, in controlling the growth of undesirable vegetation by pre-emergent or post-emergent application of said inhibitor to the plant or the locus of said vegetation.

Advantageously, as demonstrated by the method of the present invention, compounds having uracil biosynthesis inhibitory activity are also useful as herbicidal agents. The present invention encompasses herbicidally active uracil biosynthesis inhibitors identified by the method of the prensent invention such as pyrazolinones, wherein a pyrazolinone having the structure of formula I and formula Ia is preferred. Accordingly, the present invention provides a method for the use of a uracil biosynthesis inhibitor as a herbicide such as a pyrazolinone wherein a pyrazolinone having the structure of formula I and formula Ia is preferred, for the control of monocotyledenous or dicotyledenous plant species.

Formula (I) comprises the following structure:

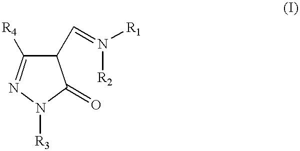

where the substituents and the index have the following meanings:

R₁ is hydrogen or C₁-C₆-alkyl unsubstituted or substitued with one to three of the following radicals: cyano, nitro, hydroxyl, mercapto, amino, carboxyl, C₁-C₄-alkoxy, C₁-C4-haloalkoxy, C₁-C₄-alkoxycarbonyl or C₁-C₆-alkylthio;

R₂ is hydroxy, OR₅, aryl, arylmethylene, hetaryl, hetarylmethylene unsubstituted or substitued with one to three of the following groups: halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, and C₁-C₆-alkoxycarbonyl;

R₃ is C₁-C₄-alkyl or hydrogen;

R₄ is hydrogen or hydroxy; and

R₅ is C₁-C₆-alkylcarbonyl or a formyl-moiety bonded to the structure via the carbon atom;

with the proviso that if R₁ is H, R₂ can also be HNR₆, wherein R₆ is aryl, arylmethylene, hetaryl, hetarylmethylene unsubstituted or substituted with halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, or C₁-C₆-alkoxycarbonyl;

or an agriculturally acceptable salt or ester thereof.

In the definitions of the formula I and Ia given above, collective terms were used which are generally representative of the following groups:

halogen: fluorine, chlorine, bromine and iodine;

alkyl: straight-chain or branched alkyl groups having 1 to 4, 6 or 10 carbon atoms, eg. C₁-C₄-alkyl such as methyl, ethyl, propyl, 1-methylethyl, butyl, 1-methylpropyl, 2-methylpropyl, 1,1-dimethylethyl or C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

aryl or arylmethylene: aromatic mono- or polycyclic hydrocarbon radicals which are bonded to the structure directly (aryl) or via an methylene group (—CH₂—) eg. phenyl, naphthyl and phenanthrenyl or phenylmethyl, naphthylmethyl or phenanthrenylmethyl;

C₃-C₆ cycloalkyl: monocyclic alkyl groups having 3 to 6 carbon ring members, eg. cyclopropyl, cyclobutyl, cyclopentyl and cyclohexyl;

hetaryl or hetarylmethylene: aromatic mono- or polycyclic radicals which, in addition to carbon ring members, additionally can contain one to four nitrogen atoms or one to three nitrogen atoms and an oxygen and/or a sulfur atom and/or an oxygen which are bonded to the structure directly or via a methylene group (—CH₂—)

5-membered heteroaryl, containing one to four nitrogen atoms or one to three nitrogen atoms and a sulfur or oxygen atom or an oxygen or a sulfur atom: 5-membered ring heteroaryl groups which, in addition to carbon atoms, can contain one to four nitrogen atoms or one to three nitrogen atoms and a sulfur or oxygen atom or an oxygen or sulfur atom as ring members, eg. 2-furyl, 3-furyl, 2-thienyl, 3-thienyl, thiophen-2-yl, thiophen-3-yl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl, 1,3,4-triazol-2-yl;

6-membered heteroaryl, containing one to three or one to four nitrogen atoms: 6-membered ring heteroaryl groups which, in addition to carbon atoms, can contain one to three or one to four nitrogen atoms as ring members, eg. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetra-zinyl;

alkylcarbonyl: straight-chain or branched alkyl groups having 1 to 4 carbon atoms, which are bonded to the structure via a carbonyl group (—CO—);

haloalkyl: straight-chain or branched alkyl groups having 1 to 4 carbon atoms, it being possible for the hydrogen atoms in these groups to be partly or completely replaced by halogen atoms as mentioned above, eg. C₁-C₂-haloalkyl such as chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

C₃-C₅ alkylen: straight-chain alkylenegroups of the general formula —(CH₂)ₙ—, wherein n=3,4 or 5;

haloalkoxy: straight-chain or branched alkyl groups having 1 to 6 carbon atoms, it being possible for the hydrogen atoms in these groups to be partly or completely replaced by halogen atoms as mentioned above, and these groups being bonded to the structure via an oxygen atom;

dialkylsulfonamino: Sulfonamino-group bonded to the structure via the S-atom, wherin the amino-group carries two C₁-C₄ alkyl groups;

alkoxycarbonyl: straight-chain or branched alkyl groups having 1 to 4 carbon atoms, which are bonded to the structure via an oxycarbonyl group (—OC(═O)—);

cycloalkyl: monocyclic alkyl groups having 3 to 6 carbon ring members, eg. cyclopropyl, cyclobutyl, cyclopentyl and cyclohexyl;

alkylamino: an amino group which carries a straight-chain or branched alkyl group having 1 to 6 carbon atoms as mentioned above;

dialkylamino: an amino group which carries two straight-chain or branched alkyl groups which are independent of one another and each have 1 to 6 carbon atoms as mentioned above;

alkylaminocarbonyl or aminocarbonyl: alkylamino or amino groups having 1 to 6 carbon atoms as mentioned above, which are bonded to the structure via a carbonyl group (—CO—);

alkylaminothiocarbonyl and aminothiocarbonyl: alkylamino or amino groups having 1 to 6 carbon atoms as mentioned above, which are bonded to the structure via a thiocarbonyl group (—CS—);

Preferably, R₄ is H (formula Ia). In another preferred embodiment, R₄ is H and R₃ is H.

More preferred are compounds of forumla I and Ia, wherein the substituents have the following meanings:

R₁ is hydrogen or C₁-C₆-alkyl it being possible for the hydrocarbon radicals of these groups to be partly or completely halogenated or to carry one to three of the following radicals: cyano, nitro, hydroxyl, mercapto, amino, carboxyl, C₁-C₄-alkoxy, C₁-C4-haloalkoxy, C₁-C₄-alkoxycarbonyl or C₁-C₆-alkylthio;

R₂ is aryl, arylmethylene, hetaryl, hetarylmethylene it being possible for the cyclic radicals in turn to be partly or completely halogenated and/or to carry one to three of the following groups: cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, C₁-C₆-alkoxycarbonyl;

R₃ is methyl or hydrogen; and

R₄ is hydrogen;

If R₁ is H, tautomers of formula I are also encompassed by the present invention, like those set forth below in sheme 1:

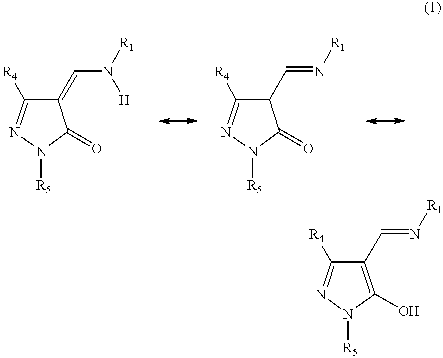

Compounds of formula Ia have been prepared using a pyrazolone of formula 4 in a five step synthesis which i.a. requires methylation and imine formation. Some of the compounds of formula I are also commercial available e.g. from Bionet Research, Ltd. Camelford, Cornwell, UK.

The present invention provides also a process for preparing the compounds (including agriculturally acceptable salts and esters thereof) of the formula Ia,

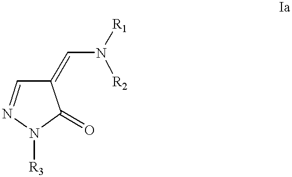

wherein the substituents R₁, R₂ and R₃ have the meaning given in formula I which comprises reacting an amoniumsalt of the formula II

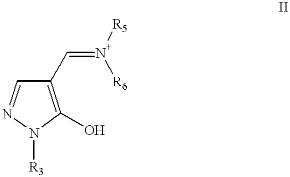

wherein the substituent R₃ has the meaning given in formula I and R₅ and R₆ both independently stand for hydrogen or C₁-C₄-alkyl or together for C₃-C₅ alkylen,

in a commonly known addition-elimination reaction manner known per se with an amine of formula III

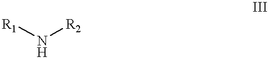

wherein the substituents R₁ and R₂ have the meaning given in formula I to give Ia.

Suitable anions are for example halogenanions like chloride, bromide, iodide or fluoride. For financial reasons, chloride is preferred.

The preparation is e.g. carried out in an organic solvent like C₁-C₄-alkohol, preferably ethanol in the absence of water.

The present invention claims compounds of the formula II and a process for preparation of said compounds based on the known mechanism of Vilsmeyer reaction which comprises reacting a pyrazolone of the formula IV (preparation of pyrazolones of formula IV is described in Angew. Chem. 18 (1966), 665-666, Engl. Ed.)

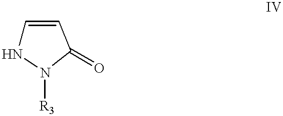

wherein R₃ has the meaning given in formula I.

solubilized or dispered in a solution of a dialkylformamide of formula V

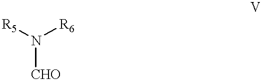

wherein the substituents R₅ and R₆ have the meaning given in formula II in the presence of a catalyst like poshorous oxy chloride (POCl₃) to give IV and work up with an C₁-C₄ alkohol, preferably ethanol wherein C₁-C₄ has the meaning given above. This isolation technique avoids an aqueous work-up and provides the key intermediate II.

To prepare pyrazolones of the formula IV e.g. propynoicacid ethyl ester of formula VI is reacted with an hydrazine of formula VII in a organic solvent like ethanol (see sheme 2), wherein R3 has the meaning given in formula I.

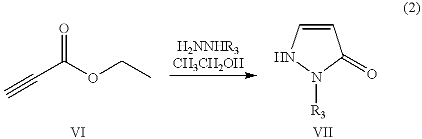

For the control of monocotyledenous or dicotyledenous plant species the following compounds identified by the method of the present invention are suitable: compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” (see pages 3-4).

Accordingly, a method for control of undesirable monocotyledenous or dicotyledenous plants which comprises applying to said plants a herbicidally effective amount of a uracil biosynthesis inhibitor e.g. of pyrazolinones, more preferably of pyrazolinones having the structure of I and Ia and most preferred of a uracil biosynthesis inhibitor selected from the group comprising compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z”.

The invention further relates specifically to novel compounds that inhibit UMP biosynthesis, either directly or indirectly, and that specifically inhibit the target enzyme activities orotate phosphoribosyltransferase (OPRTase) and orotate decarboxylase (ODCase), and/or the UMP synthase enzyme, either directly or indirectly. Preferably, the invention relates to the use of a member of the pyrazolinone family as an inhibitor of the target enzyme activities orotate phosphoribosyltransferase (OPRTase) and orotate decarboxylase (ODCase), and/or the UMP synthase enzyme, either directly or indirectly. In one embodiment, the invention relates to the use of at least one member selected from the group of pyrazolinones preferably pyrazolinones of formula I and most preferably compound “A”, compound “B”, compound “C”, compound “D”, compound “E”, compound “F”, compound “G”, compound “H”, compound “I”, compound “J”, compound “K”, compound “L”, compound “M”, compound “W”, compound “O”, compound “P”, compound “Q”, compound “R”, compound “S”, compound “T”, compound “U”, compound “W”, compound “X”, compound “Y”, and/or compound “Z” to inhibit the enzyme activities orotate phosphoribosyltransferase (OPRTase) and orotate decarboxylase (ODCase), and/or the UMP synthase enzyme, either directly or indirectly.

In yet another embodiment, the present invention encompasses the use of hydrolysis, and/or break-down products, of the identified inhibitors of the present invention as inhibitors of pyrimidine biosynthesis e.g. the use of the hydrolysis and/or break-down products of pyrazolinones more preferred pyrazolinones of formula I and Ia as inhibitors of pyrimidine biosynthesis. Most preferably, the invention encompasses the use of the hydrolysis and/or break-down products of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” as inhibitors of pyrimidine biosynthesis. Most preferably, the invention encompasses the use of pyrazole aldehyde, or its salt, or acid, as an inhibitor of pyrimidine biosynthesis. In addition, the invention encompasses the use of the respective anilines of pyrazolinones, more preferably pyrazolinones of formula I, wherein pyrazolinones comprising the formula of compound “B”, compound “C”, compound “D”, compound “E”, compound “F”, or compound “G” as inhibitors of pyrimidine biosynthesis are most preferred.

One embodiment of the invention comprises herbicidal compositions of the inhibitory compounds identified by the method disclosed herein. Such compositions may comprise one or more inhibitory compounds identified by the present invention, or known herbicidal compounds, and may additionally be formulated according to the formulation methods and compositions described herein. In one embodiment, the herbicidal compositions comprises at least one member of the pyrazolinone family of compounds, more preferably of pyrazolinones of formula I. Most preferably, the herbicidal compositions comprise at least one member selected from the group consisting of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z”, or other compositions active on the uracil biosynthesis pathway.

In an alternative embodiment, the herbicidal compositions of the present invention comprise a compound selected by the methods mentioned above that is not a member of the pyrazolinone family.

Another aspect of the present invention is a method for the control of undesirable plant growth that comprises applying to a plant or a locus where control is desired a herbicidally effective amount of an inhibitory compound identified according to the method disclosed herein. In addition, the present invention encompasses a method for the control of undesirable plant growth that comprises applying to a a plant or a locus where control is desired a herbicidally effective amount of a pyrazolinone compound preferably of a pyrazolinone compound comprising formula I or formula Ia. Moreover, the present invention encompasses a method for the control of undesirable plant growth that comprises applying to a a plant or a locus where control is desired a herbicidally effective amount of at least one member selected from the group consisting of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z”, or other compositions active on this pathway. Alternatively, such a method comprises the application of a modified form of an inhibitory compound identified according to the method disclosed herein, which may include phosphorylation, reduction, oxidation, or derivitivation, for example.

In one embodiment of the present invention, the invention encompasses the use of potential hydrolysis products of the inhibitory compounds identified by the method disclosed herein as herbicides. In one embodiment of the present invention these compounds belong to the pyrazolinones, more preferred to pyrazolinones comprising formula I and Ia and most preferred of compounds belonging to the group consisting of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z”. In yet another embodiment, the present invention encompasses the use of hydrolysis, and/or break-down products, of the identified inhibitors of the present invention as herbicides. Thus, the invention encompasses the use of the hydrolysis and/or break-down products of pyrazolinones, more preferably of pyrazolinones comprising formula I and Ia as herbicides. Most preferably, the invention encompasses the use of the hydrolysis and/or break-down products of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” as herbicides. Most preferably, the invention encompasses the use of pyrazole aldehyde, or its salt, or acid, as a herbicide. In addition, the invention encompasses the use of the respective anilines of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” as herbicides.

In yet another embodiment of the present invention, the invention encompasses the use of the inhibitory compounds identified by the method disclosed herein as fungicides, nematicides, and/or insecticides.

Moreover, the present invention encompasses the application of the inventive method to highthroughput methodology. An exemplary application would be carrying out the inventive method to identify uracil biosynthesis inhibitors in microtiter plates. Such an application in encompassed by the present invention and is described elsewhere herein.

Uses for the Compounds Identified by the Method of the Present Invention

The compounds identified by the present invention are useful for inhibiting pyrimidine biosynthesis, either directly or indirectly. The compounds of the present invention may be useful for inhibiting the pyrimidine de novo biosynthesis pathway, either directly or indirectly. The compounds of the present invention may be useful for inhibiting the pyrimidine salvage biosynthesis pathway, either directly or indirectly. Specifically, the compounds of the present invention may be useful in inhibiting, either directly or indirectly, cytidine, uridine, uracil, and thymidine biosynthesis, and possibly, the biosynthesis of known pyrimidine analogs and/or variants, though are preferably useful for inhibiting uracil biosynthesis.

The compounds identified by the present invention may be useful in inhibiting pyrimidine biosynthesis through, either direct or indirect, inhibition of aspartate transcarbamoylase, dihydroorotase, dihydroorotate dehydrogenase, orotate phosphoribosyl transferase, orotidylate decarboxylase, and/or UMP synthase, though preferably UMP synthase. Moreover, the compounds indentified by the present invention may inhibit pyrimidine biosynthesis through, either direct or indirect inhibition of 5′nucleosidase, uridine kinase, uridylate kinase, nucleoside diphosphatase, nucleoside diphosphate kinase, nucleoside diphosphatase, uracil phosphoribosyltransferase, uridine nucleosidase, and/or uridine phosphorylase. In addition, the compounds of the present invention may inhibit pyrimidine biosynthesis through, either direct or indirect inhibition of aspartate, carbamoyl phosphate, N carbamoylaspartate, L-dihydroorotate, NAD+, PRPP, orotate, orotidine-5′-phosphate, and/or uridine-5′-phosphate.

The compounds identified by the methods of the present invention are useful as herbicides. The compounds are useful, for example, in controlling the growth of undesirable vegetation by pre-emergent or post-emergent application to the plant or the locus where control is desired.

Although the compounds identified by the methods of the present invention may be useful as herbicides, the skilled artisan would appreciate that such compounds may have other uses as well. Specifically, the compounds may have fungicidal, insecticidal, and/or nematicide activities. Alternatively, the compounds may have a combination of herbicidal activity and at least one of the following activities: fungicidal, insecticidal, and nematicide activities. For example, a compound identified by the methods of the present invention may have potent herbicidal UMP synthase inhibitory activity. Since other organisms have this enzyme, the compound may also be capable of inhibiting UMP synthase activity in a fungicide, insecticide, and/or nematacide capacity, for example.

The compounds identified by the method of the present invention may also be useful as a bacteriocide.

The compounds identified by the method of the present invention may be useful for inhibiting transcription, in general. Such a use would be beneficial for research purposes, particularly when used in combination with other transcription inhibitors.

The compounds identified by the method of the present invention may be useful for identifying complementation mutants of the pyrimidine biosynthesis pathway, and specifically for the de novo and salvage pyrimidine biosynthesis pathways. In one example, if a particular compound identified by the methods of the present invention specifically inhibits a particular enzyme in the pyrimidine de novo biosynthesis pathway, for example, random or directed mutants could be generated, and incubated in the presence of a compound of the present invention, and the surviving mutants isolated and characterized. The mutants that survive may represent complementation mutants of the pyrimidine de novo biosynthesis pathway. The methods of generating mutants are known in the art, but may include incubation with a chemical mutagen, such as DMS or EMS, for example. Additional methods of generating mutants may be found in Sambrook J. L., et al., Molecular cloning: A Laboratory Manual. (2nd ed.) Cold Spring Harbor Laboratory Press, Cold Spring Harbor, N.Y., (1989)).

In another example, the compounds identified by the present invention are useful for selectively studying a particular pyrimidine biosynthesis pathway. For example, if a compound identified by the methods of the present invention specifically inhibits a particular enzyme in the de novo pyrimidine biosynthetic pathway, and the sample is incubated in the presence of the inhibitor, such an inhibitor would enable selective characterization of the salvage pyrimidine biosynthetic pathway as the de novo pathway would not be functional, for example.

In yet another example, more than one compound identified by the method of the present invention may be combined to facilitate characterization of either the de novo, or salvage pyrimidine biosynthetic pathways, or both, in addition to facilitating the possible isolation of complementation mutants of either or both pathways.

The compounds identified by the method of the present invention are useful as a reagent in a diagnostic kit. For example, a compound of the present invention may inhibit a particular enzyme in a pathway for pyrimidine biosynthesis. Since the particular enzyme may exist in several allelic forms, the compound may inhibit only one particular allelic form, thus enabling rapid identification of which form of the enzyme a particular plant is expressing or responsible for a particular phenotype or trait, for example. Alternatively, specific mutations may exist in the general plant populations that are either sensitive or resistant to the compound of the present invention. Therefore, rapid identification of the plants harboring the mutant or wild type form of the enzyme may be possible.

Such diagnostic kits may comprise a suitable support for holding a sample (preferably a solid support), and a test compound. The sample may be any plant, plant tissue, or plant cell, in addition to other plant tissues described herein and known in the art.

The present invention further encompasses a process for production of pesticidal agents comprising the following steps:

a) identifying uracil biosynthesis inhibitor by the method of the present invention; and

b) preparing a pesticidal composition comprising the pesticidal agent identified in step a).

The term “formulation” means that Uracil biosynthesis inhibitor compounds can be applied as a spray, a powder, a granule, or in any of the other conventional methods known in the agricultural art. The invention provides a herbicidal composition that comprises an inert carrier and a herbicidally effective amount of a uracil biosynthesis inhibitor such as pyrazolinones, pryrazolinones of formula I and Ia, wherein compounds belonging to the group consisting of compound “A”, compound “B”, compound “C”, compound “D”, compound “F”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” are preferred. In yet another embodiment the herbicidal composition comprises at least a herbicidally effective amount of a break down or hydrolysis products of an uracil biosynthesis inhibitor such as such as pyrazolinones, pryrazolinones of formula I and Ia, wherein compounds belonging to the group consisting of compound “A”, compound “B”, compound “C”, compound “D”, compound “E”, compound “F”, compound “G”, compound “H”, compound “I”, compound “J”, compound “K”, compound “L”, compound “M”, compound “N”, compound “O”, compound “P”, compound “Q”, compound “R”, compound “S”, compound “T”, compound “U”, compound “W”, compound “X”, compound “Y”, and/or compound “Z” are preferred.

The specific formulation used will depend on a variety of factors unique to the chemical compounds themselves, the specific environmental conditions (e.g., temperature, pH, humidity, etc.), the intended purpose of the formulation (e.g., to facilitate dispersion, stabilize the chemical compounds, increase adhesion properties, etc.) and/or the characteristics of the recipient organism of the chemical compound. Due to these varied factors, the choice of formulation and mode of application for any given compound may affect its activity, and selection may be made accordingly.

The uracil biosynthesis inhibitors such as a compound of I and Ia may be formulated as a wettable powder, an emulsifiable concentrate, a powder or dust, a flowable, a solution, a suspension, an emulsion, a controlled-release composition such as a microcapsule, or any of the conventional forms known to be suitable for agricultural applications. The composition of the invention may contain about 0.5% to about 95% by weight of a uracil biosynthesis inhibitor. The optimum amount for any given compound will depend upon the nature of the plant to be controlled. The rate of application may vary from about 0.01 to about 11.5 kilograms per hectare, preferably from about 0.02 to about 4.5 kilograms per hectare.

Wettable powders are in the form of finely divided particles which disperse readily in water or other liquid carriers. The particles contain the active ingredient retained in a solid matrix. Typical solid matrices include fuller's earth, kaolin clays, silicas and other readily wettable organic or inorganic solids. Wettable powders normally contain about 5% to about 95% of the active ingredient plus a small amount of wetting, dispersing, or emulsifying agent.

Emulsifiable concentrates are homogeneous liquid compositions dispersible in water or other liquid, and may consist entirely of the active compound with a liquid or solid emulsifying agent, or may also contain a liquid carrier, such as xylene, heavy aromatic naphthas, isophorone and other nonvolatile organic solvents. In use, these concentrates are dispersed in water or other liquid and normally applied as a spray to the area to be treated. The amount of active ingredient may range from about 0.5% to about 95% of the concentrate.

Dusts are free-flowing admixtures of the active ingredient with finely divided solids such as talc, clays, flours and other organic and inorganic solids which act as dispersants and carriers.

Microcapsules are typically droplets or solutions of the active material enclosed in an inert porous shell that allows escape of the enclosed material to the surrounds at controlled rates. Encapsulated droplets are typically about 1 to 50 microns in diameter. The enclosed material typically constitutes about 50 to 95% of the weight of the capsule, and may include solvent in addition to the active compound. Shell of membrane materials include natural and synthetic rubbers, cellulosic materials, styrene-butadiene copolymers, polyacrylonitriles, polyacrylates, polyesters, polyamides, polyureas, polyurethanes and starch xanthates.

Compositions of the invention include simple solutions of the active ingredient in a solvent in which it is completely soluble at the desired concentration, such as water, acetone, alkylated naphthalenes, xylene and other organic solvents. Pressurized sprayers, wherein the active ingredient is dispersed in finely-divided form as a result of vaporization of a low boiling dispersant solvent carrier may also be used.

Inert carriers suitable for use in the composition of the invention include wetting, dispersing or emulsifying agents. Examples are alkyl and alkylaryl sulfonates and sulfates and their salts; polyhydric alcohols; polyethoxylated alcohols; esters and fatty amines. These agents, when used, may comprise from about 0.1% to about 15% by weight of the inventive composition.

The compounds identified by the method of the present invention are also useful when combined with other active compounds (e.g., herbicides, fungicides, insecticides, etc.) and/or defoliants, desiccants, growth inhibitors, and the like. These other materials can comprise from about 5% to about 95% of the active ingredients in the formulations. These combinations frequently provide a higher level of effectiveness in controlling weeds and often provide results unattainable with separate formulations of the individual active compounds.

Such formulations may also contain more than uracil biosynthesis inhibitor, though preferably at least one, two, three, four, or more. The purpose for combining more than one active compound identified by the method of the present invention may be to take advantage of each compounds unique characteristics. For example, one compound may have very high activity, though may be inactive in the presence of water (e.g., rain, etc.), while another compound, for example, may have less activity, though is more resistant to the presence of water. Thus, a combination of both compounds would ensure improved efficacy under a variety of conditions.

Examples of other compounds, defoliants, desiccants and plant growth inhibitors with which the uracil biosynthesis inhibitors may be combined are: Benzo-2,1,3-thiadiazin4-one-2,2-dioxides such as bentazone; hormone herbicides, particularly the phenoxyalkanoic acids such as MCPA, MCPA-thioethyl, dichlorprop, 2,4,5-T, MCPB, 2,4-D, 2,4-DB, mecoprop, trichlopyr, fluroxypyr, clopyralid, and their derivatives (e.g. salts, esters and amides); pyrazole derivatives such as pyrazoxyfen, pyrazolate and benzofenap; dinitrophenols and their derivatives (e.g. acetates such as DNOC, dinoterb, dinoseb and its ester, dinoseb acetate; dinitroaniline herbicides such as dinitramine, trifluralin, ethalfluralin, pendimethalin; and oryzalin; arylurea herbicides such as diuron, flumeturon, metoxuron, neburon, isoproturon, chlorotoluron, chloroxuron, linuron, monolinuron, chlorobromuron, daimuron, and methabenzthiazuron; phenylcarbamoyloxyphenylcarbamates such as phenmedipham and desmedipham; 2-phenylpyridazin-3-ones such as chloridazon, and norflurazon; uracil herbicides such as lenacil, bromacil and termacil; triazine herbicides such as atrazine, simazine, aziprotryne, cyanazine, prometryn, dimethametryn, simetryne, and terbutryn; phosphorothioate herbicides such as piperophos, bensulide, and butamifos; thiolcarbamate herbicides such as cycloate, vemolate, molinate, thiobencarb, butylate, EPTC, triallate, diallate, ethyl esprocarb, tiocarbazil, pyridate, and dimepiperate (the butylate and EPTC compounds are preferably employed in combination with a safener such as 2,2-dichloro-N,N-di-2-propenylacetamide (dichlormid)); 1,2,4-triazin-5-one herbicides such as metamitron and metribuzin; benzoic acid herbicides such as 2,3,6-TBA, dicamba and chloramben; anilide herbicides such as pretilachlor, butachlor, the corresponding alachlor, the corresponding compound propachlor, propanil, metazachlor, metolachlor, acetochlor, and dimethachlor; dihalobenzonitrile herbicides such as dichlobenil, bromoxynil and ioxynil; haloalkanoic herbicides such as dalapon, TCA and salts thereof; diphenylether herbicides such as lactofen, fluroglycofen or salts or esters thereof, nitrofen, bifenox, acifluorfen and salts and esters thereof, oxyfluorfen and fomesafen; chlornitrofen and chlomethoxyfen; phenoxyphenoxypropionate herbicides such as diclofop and esters thereof such the methyl ester, fluazifop and esters thereof, haloxyfop and esters thereof, quizalofop and esters thereof and fenoxaprop and esters thereof such as the ethyl ester; triketone and cyclohexanedione herbicides such as alloxydim and salts thereof, sethoxydim, cycloxydim, sulcotrione, tralkoxydim, and clethodim; sulfonyl urea herbicides such as chlorosulfuron, sulfometuron, metsulfuron and esters thereof; benzsulfuron and esters thereof such as the ester thereof methyl, DPX-M6313, chlorimuron and esters such as the ethyl ester thereof, pirimisulfuron and esters such as the methyl ester thereof, DPX-LS300 and pyrazosulfuron; imidazolidinone herbicides such as imazaquin, imazamethabenz, imazapyr and isopropylammonium salts thereof, imazathapyr; arylanilide herbicides such as flamprop and esters thereof, benzoylprop-ethyl, diflufenican; amino acid herbicides such as glyphosate and gluyfosinate and their salts and esters, sulphosate, and bilanafos; organoarsenical herbicides such as MSMA; herbicidal amide derivative such as napropamide, propyzamide, carbetamide, tebutam, bromobutide, isoxaben, naproanilide, diphenamid, and naptalam; 4-benzoylisoxazole and 2-cyano-1,3-dione herbicides; miscellaneous herbicide compounds include ethofumesate, cinmethylin, difenzoquat and salts thereof such as the methyl sulfate salt, clomazone, oxadiazon, bromofenoxim, barban, tridiphane, flurochloridone, quinchlorac and mefanacet; contact herbicides, examples of which include bipyridylium herbicides such as those in which the active entity is paraquat and those in which the active entity is diquat.

Additionally, the uracil biosynthesis inhibitors may also be mixed with one or more other insecticides, fungicides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection. Examples of such agricultural protectants with which compounds identified by the method of the present invention can be formulated are: insecticides such as abamectin, acephate, azinphos-methyl, bifenthrin, buprofezin, carbofuran, chlorpyrifos, chlorpyrifos-methyl, cyfluthrin, beta-cyfluthrin, deltamethrin, diafenthiuron, diazinon, diflubenzuron, dimethoate, esfenvalerate, fenpropathrin, fenvalerate, fipronil, flucythrrinate, tau-fluvalinate, fonophos, imidacloprid, isofenphos, malathion, metaldehyde, methamidophos, methidathion, methomyl, methoprene, methoxychlor, monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; fungicides such as azoxystrobin (ICIA5504), benomyl, blasticidin-S, Bordeaux mixture (tribasic copper sulfate), bromuconazole, captafol, captan, carbendazim, chloroneb, chlorothalonil, copper oxychloride, copper salts, cymoxanil, cyproconazole, cyprodinil (CGA 219417), diclomezine, dicloran, difenoconazole, dimethomorph, diniconazole, diniconazole-M, dodine, edifenphos, epoxyconazole (BAS 480F), fenarimol, fenbuconazole, fenpiclonil, fenpropidin, fenpropimorph, fluquinconazole, flusilazole, flutolanil, flutriafol, folpet, fosetyl-aluminum, furalaxyl, hexaconazole, ipconazole, iprobenfos, iprodione, isoprothiolane, kasugamycin, kresoxim-methyl (BAS 490F), mancozeb, maneb, mepronil, metalaxyl, metconazole, myclobutanil, neo-asozin (ferric methanearsonate), oxadixyl, penconazole, pencycuron, probenazole, prochloraz, propiconazole, pyrifenox, pyroquilon, sulfur, tebuconazole, tetraconazole, thiabendazole, thiophanate-methyl, thiram, triadimefon, triadimenol, tricyclazole, triticonazole, uniconazole, validamycin and vinclozolin; nematocides such as aldoxycarb and fenamiphos; bactericides such as streptomycin; acaricides such as amitraz, chinomethionat, chlorobenzilate, cyhexatin, dicofol, dienochlor, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi.

Preferably, the combinations described above are specifically chosen to include compounds that have a similar spectrum of control but a different mode of action as such combinations would be be particularly advantageous for resistance management.

The composition of the invention may be applied to the areas where control is desired by a variety of conventional methods well known in the art. Specifically, dust and liquid compositions, for example, can be applied by the use of powerdusters, boom and hand sprayers and spray dusters. The formulations can also be applied from airplanes as a dust or a spray or by rope wick applications.

The following are non-limiting examples of typical formulations with the final weight percentage totaling 100%. The unit % generally stands for wt %.

Dusts:

5% dust: 5 parts active compound 95 parts talc

2% dust: 2 parts active compound 1 part highly dispersed silicic acid 97 parts talc

These dusts are formed by mixing the components then grinding the mixture to the desired particle size.

Wettable powders:

70%: 70 parts active compound 5 parts sodium dibutylnaphthylsulfonate 3 parts naphthalenesulfonic acid/phenolsulfonic acidlphenol-sulfonicacid/formaldehyde condensate (3:2:1) 10 parts kaolin 12 parts Champagne chalk

40%: 40 parts active compound 5 parts sodium lignin sulfonate 1 part sodium dibutylnaphthalene sulfonic acid 54 parts silicic acid

25% 25 parts active compound 4.5 parts calcium lignin sulfate 1.9 parts Champagne chalk/-hydroxyethyl cellulose (1:1) 8.3 parts sodium aluminumsilicate 16.5 parts kieselguhr 46 parts kaolin

10% 10 parts active compound 3 parts of a mixture of sodium salts of saturated fatty alcohol sulfates 5 parts naphthalenesulfonic acid/formaldehydecondensate 82 parts kaolin

These wettable powders are prepared by intimately mixing the active compounds with the additives in suitable mixers, and grinding the resulting mixture in mills or rollers.

Emulsifiable concentrate:

25%: 25 parts active substance 2.5 parts epoxidized vegetable oil 10 parts of an alkylarylsulfonate/fatty alcohol polyglycol ether mixture 57.5 parts xylene.

The amount of the present compositions which constitute a herbicidally effective amount depends upon the nature of the seeds or plants to be controlled. The rate of application of active ingredients varies from about 0.01 to about 28 kilograms per hectare, preferably about 0.02 to about 11 kilograms per hectare with the actual amount depending on the overall costs and the desired results. It will be readily apparent to one skilled in the art that compositions exhibiting lower herbicidal activity will require a higher dosage than more active compounds for the same degree of control.

Moreover, the present invention encompasses testing plants, plant cells, plant tissues, and/or plant seeds for uracil biosynthesis inhibition, herbicidal effects, fungicidal effects, nematicidal effects, and/or arthropod inhibitory effects, for the compounds identified by the method of the present invention. The invention also encompasses the use of any plant tissue, plant cell, or plant seed.

Rates of application for these compounds can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

The present compositions may be prepared in a known manner, for example by homogeneously mixing or grinding the active ingredient(s) with other ingredients. Additional components may be admixed with the composition at any point during the process, including during and/or after any mixing step of the herbicide components.

Experimental:

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, percent is percent by weight given the component and the total weight of the composition, temperature is in ° C. or is at ambient temperature, and pressure is at or near atmospheric.

### EXAMPLES

### Example 1

### Determination of Herbicidal Effects of Probe Compounds

The herbicide efficacy of the pyrazolinones both preemergence and post-emergence was evaluated on various weed species. Table 1 summarizes the effect of probe compound on whole plant injury. As illustrated, the probe compound has a herbicidal effect of 50% or more injury especially on grass species, with safety on wheat with preemergence and post-emergence applications. These results illustrate the herbicidal activity of the tested probe compound.

Methods:

The compounds were applied to flats containing individually potted plant species as post-emergent treatments, or seeded flats without emerged plants for preemergent treatments. The compounds were delivered in an 80% acetone solution plus 0.2% X-77. After two or three weeks of treatment, plants were rated compared to controls on a scale of 0 to 9 where 0 represents no injury and 9 represents 100% injury.

### Example 2

### Further Evaluation of the Herbicidal Activity of the Probe Compound

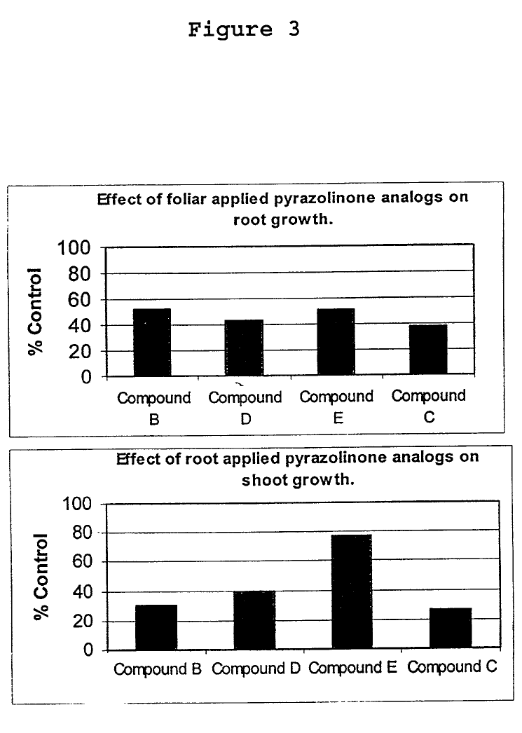

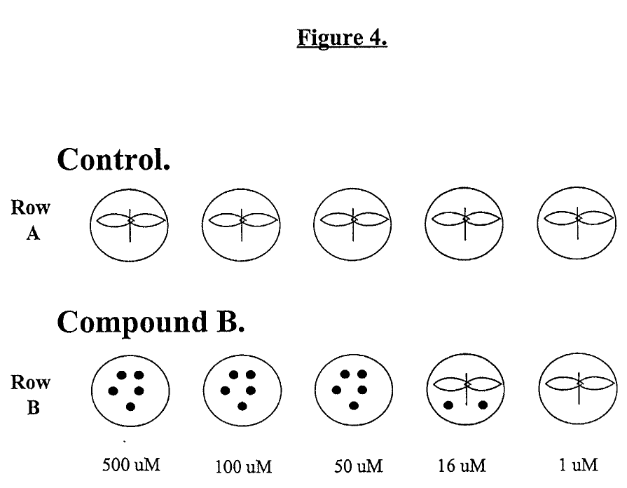

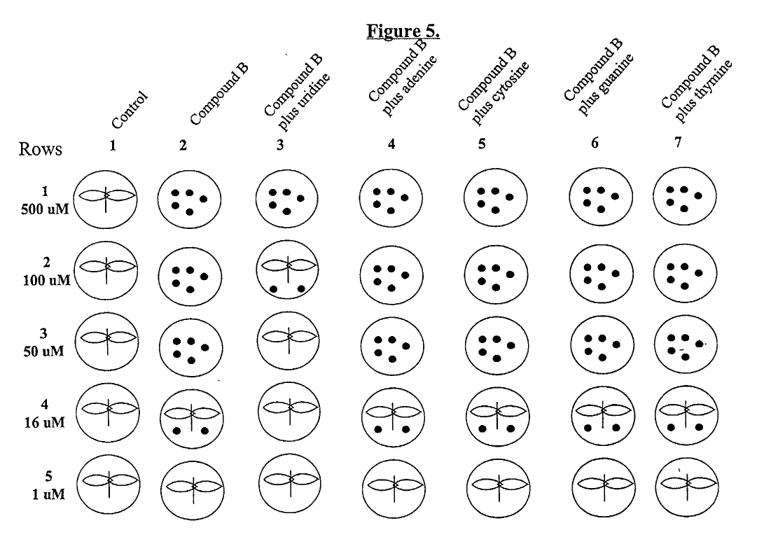

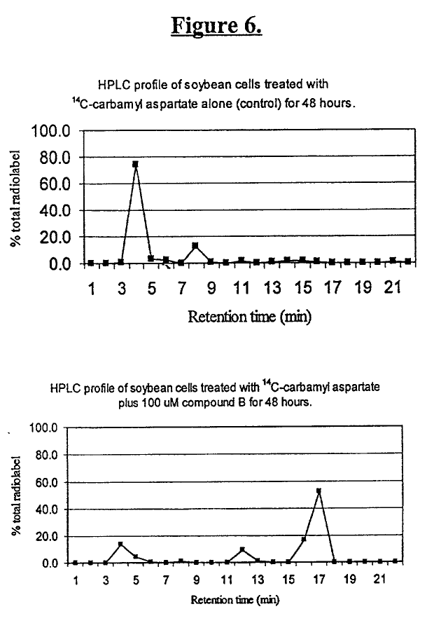

Hydroponic tests provide a useful way of determining if compounds are phloem or xylem mobile in a plant system. As illustrated in FIG. 3, the analogs of a probe compound were more xylem mobile than phloem mobile. These results provide further evidence to support that the probe compounds are herbicidal and mobile within the plant system.

Methods:

Corn plants were grown in controlled-environment growth chambers (28/20 □C. day/night, 300 E/m2/sec light intensity, 14-hr photoperiod) for one week. Seedlings were germinated in vertical tubes of wet toweling in the growth chamber. The germinated seedlings were then transplanted into glass bottles and grown in Hoagland's nutrient (25 mL) solution. Seedlings were treated with the probe compound at the first trifoliate leaf stage. Probe compound was applied to the Hoagland nutrient solution for root applications so the final concentration in 25 mL was 50-100 μM, or by applying 6 uL of a solution to the oldest leaf for foliar treatments. After 5 days of treatment injury (stunting and chlorosis) was observed. Plants were harvested and root and shoot fresh weights are recorded.

### Example 3

### Determination of a Lethal Concentration of Tested Probe Compound and Reversal of Injury Symptoms

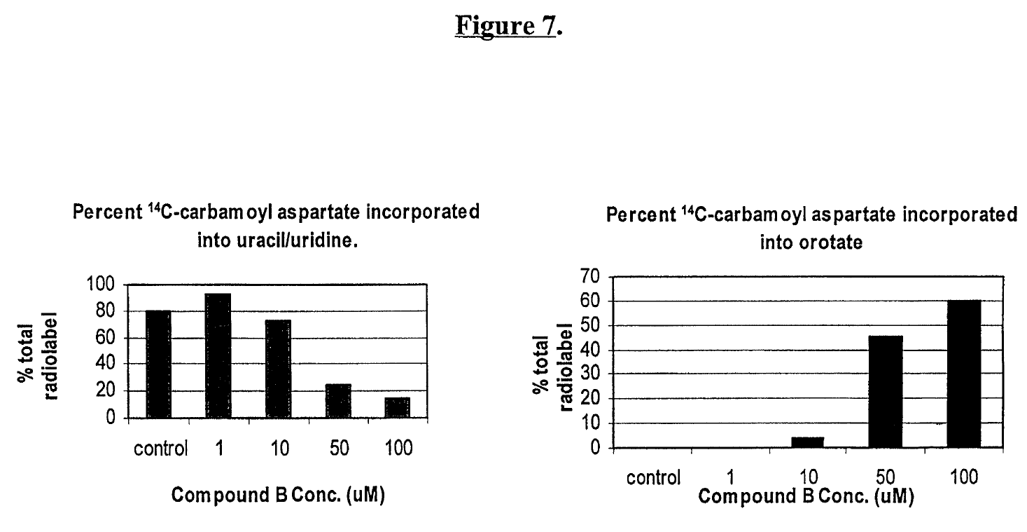

Compounds were tested at different concentrations to assess the herbicidal activity on Arabidopsis in a 96 well microtiter plate. The probe compound was herbicidal at a wide range of concentrations, causing severe stunting and chlorosis of the seedlings, which ultimately leads to plant death (Table 2 and FIG. 4). The estimated IC₅₀ for the probe compound on Arabidopsis was 30 uM.

The injury symptoms induced by the probe compound were reversed by the addition of 100 uM uracil, UMP, and uridine to the growth media (Table 2, FIG. 5). For example, at 30 uM, the probe compound alone caused 50% growth inhibition, but when combined with 100 uM uracil there was 0% growth inhibition.

Uracil, uridine, and UMP were the only compounds that reversed the herbicidal activity of the probe compound. Neither vitamins nor purines reversed the herbicidal effects of compound “B”. These results provide evidence to suggest that the probe compound specifically inhibits pyrimidine biosynthesis in vivo, and that inhibition of uracil biosynthesis is herbicidal. Orotate or OMP, intermediates of the UMP de novo pathway, did not reverse the herbicidal effects of the probe compound further suggesting that inhibition occurs in the de novo UMP biosynthesis pathway.

Cytotoxicity of certain pharmaceutical inhibitors on animal cells that target the uracil biosynthesis pathway can be reversed by the addition of uridine (Cornelius, T. B. et al. (1981) J. of Cell. Physiol. 107:335-344). However, there is no mention in the literature of using uracil or pyrimidines as reversal agents for detecting herbicidal compounds that target the uracil biosynthesis pathway. Reversal studies have been used for detection of inhibitors in other pathways in plants. For example, the effect of hydantocidin herbicidal activity, which targets the purine biosynthesis pathway, can be reversed by the addition of certain purines to the growth media (Siehl, D. L. et al. (1996) Plant Physiol. 110:753-758, and U.S. Pat. No. 5,780,254, 1996).

Reversal of herbicidal injury on Arabidopsisby uracil provides a novel method for screening of herbicidal compounds that target the UMP biosynthesis pathway in a high-throughput manner. Table 3 summarizes the effect of various compound analogs on activity (and reversal), greenhouse inhibition activity, and corn injury. Table 4 summarizes the effect of other compound analogs on Arabidopsis inhibition (and reversal) in a micotiter plate format, and greenhouse inhibition activity.

Methods:

The compounds were tested in a 96 microtiter well plate containing various concentrations of probe compound with or without intermediates of the pyrimidine and purine biosynthesis pathways to detect reversal of injury. Plates were filled with 0.7% agar medium, seeded with Arabidopsis, and placed in a growth chamber for several days to allow for seed germination and plant growth. Arabidopsis plates were rated on a scale from 0 to 9 where 0 represents no injury, and 9 represents no germination. In addition, injury symptoms observed were recorded.

### Example 4

### Determination of the Inhibitory Effect of Probe Compound on UMP Biosynthesis

As a method to further evaluate the mode of action of a probe compound and its analogs, the effect of probe compound on the incorporation of [¹⁴C]-carbamoyl aspartate into uracil was evaluated (FIG. 6). Carbamoyl aspartate is the first intermediate of the UMP biosynthesis pathway. Monitoring the incorporation of radiolabled pathway intermediates into nucleotides is a common method used for studying the inhibitory effect of compounds. In plants, these methods have often been used to study nutrient deficiency (Ashihara, H. et al. (1988). Ann. of Botany 61:225-232.). In the pharmaceutical area, where specific inhibitors of OPRTase/ODCase were studied, these methods were used to illustrate that inhibitors of uracil biosynthesis cause the nucleotide pools to decrease, and orotate levels to increase when monitoring the incorporation of [¹⁴C]-bicarbonate (Seymour, K. K. et al. (1994) Biochem. 33:5268-5274; Cornelius, T. B. et al. (1981) J. of Cell. Physiol. 107:335-344).

HPLC analysis of the soluble fraction obtained from soybean cells preincubated with radioisotope and inhibitor revealed that there were differences of [¹⁴C]-carbamoyl aspartate incorporation into uracil between control and the probe compound (FIG. 6). In control, 78% [¹⁴C]-carbamoyl aspartate was incorporated into uracil and/or uridine, and 13% had a retention time of 7-9 min, which could correspond to unincorporated carbamoyl aspartate. In cells treated with probe compound, there was only 19% incorporation of [¹⁴C]-carbamoyl aspartate into uracil/uridine, and most (70%) of the substrate was incorporated into a peak with a retention time of 16-19 min corresponding to orotate. The orotate peak was also seen under UV light in the HPLC spectrum, and its identity was confirmed by mass spectrometry. The orotate peak was not detected in control. This HPLC profile difference between control and probe compound was observed at 24, 48, and 72 hours after treatment. The differential incorporation of [¹⁴C]-carbamoyl aspartate between control and compound “B” treated soybean cells was not due to differences in uptake of [¹⁴C]-carbamoyl aspartate.

The in vivo inhibition of [¹⁴C]-carbamoyl aspartate incorporation by probe compound was concentration dependent (FIG. 7). With increasing concentration of probe compound there was less incorporation of [¹⁴C]-carbamoyl aspartate into uracil/uridine, and increasing accumulation of orotate. The concentration at which there was 50% inhibition (IC₅₀) is estimated to be approximately 50 uM, similar to the IC₅₀ value detected in the miniscreen studies with Arabidopsis. These results further provide evidence to suggest that the probe compound inhibits the incorporation of [¹⁴C]-carbamoyl aspartate into uracil/uridine in vivo in soybean cell cultures, and that inhibition may occur between orotate and its conversion to UMP.

Methods:

Soybean suspension cultures in the linear growth phase were used for [¹⁴C]carbamoyl aspartate uptake and incorporation studies. Cells were treated with 0.01 uM [¹⁴C]carbamoyl aspartate (approximately 200,000-400,000 dpm) with or without 100 uM compound “B”. Over time, aliquots were harvested by filtration, and rinsed thoroughly with potassium phosphate buffer, pH 3.11. Aliquots of uptake media and rinse were counted using a liquid scintillation spectrophotometer to determine percent uptake. Cells were immediately weighed, ground to a fine powder in liquid nitrogen, and suspended in 50% methanol. The homogenate was centrifuged at 10,000 g for 20 min. A portion of the pellet was weighed out and combusted to determine dpm's associated with this fraction. An aliquot of the supernatant was counted to determine the amount of label in the soluble fraction. Recovery of applied radiolabeled [¹⁴C]carbamyol aspartate was 90-100%.

The soluble supernatant was concentrated overnight by lyophilization, and the extract was resuspended in 1 ml potassium phosphate buffer, pH 3.11. For HPLC analysis an anion exchange column was used with 85:15% potassium phosphate buffer, pH 3.11 and acetonitrile as running buffers. Fractions (1 ml) were collected for 20 min (flow rate 1 mmin), and counted using a liquid scintillation counter (LSC). UV absorbance was analyzed at 260 and 275 nm as the optimum absorbency wavelengths for UMP and orotate, respectively. Standards were used to identify the various peaks from the HPLC profile. Standard retention times under these conditions were: 3-4 min uracil/uridine, 7-9 min carbamoyl aspartate, 11-13 min UMP, and 17-19 min orotate.

### Example 5

### Determination of the Inhibitory Effect on Enzyme Activity by Probe Compound

Orotate phosphoribosyl transferase/Orotate decarboxylase (OPRTase/ODCase).

The in vivo accumulation of orotate with probe compound and [¹⁴C]-carbamoyl aspartate suggested that uracil biosynthesis inhibition was occurring at the OPRTase/ODCase enzyme in the uracil biosynthesis pathway. In vitro enzyme studies were therefore conducted to test this possibility.

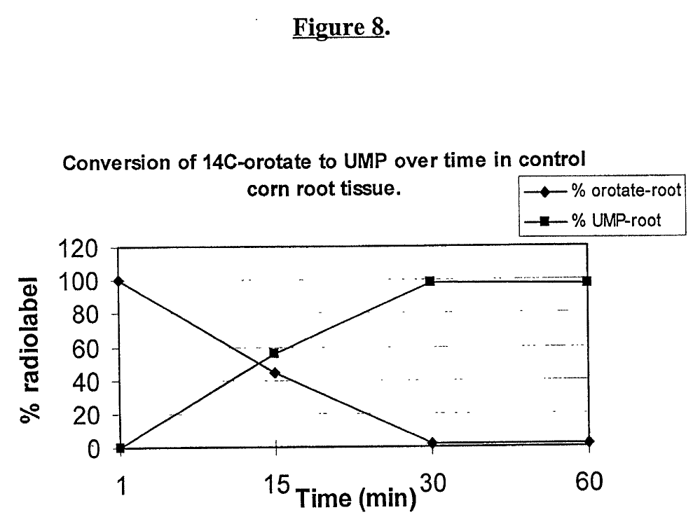

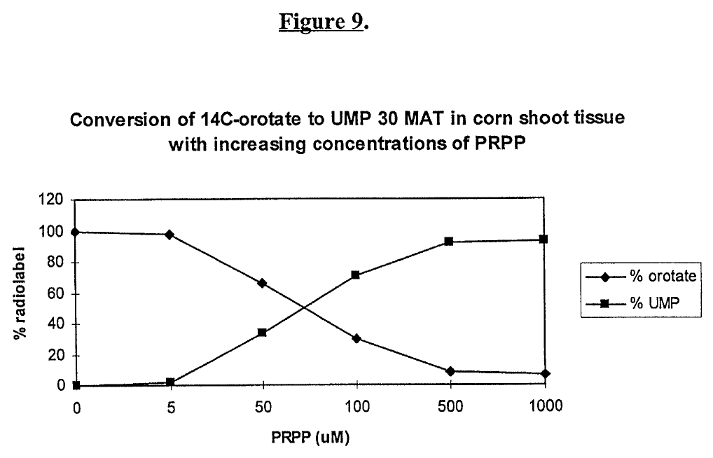

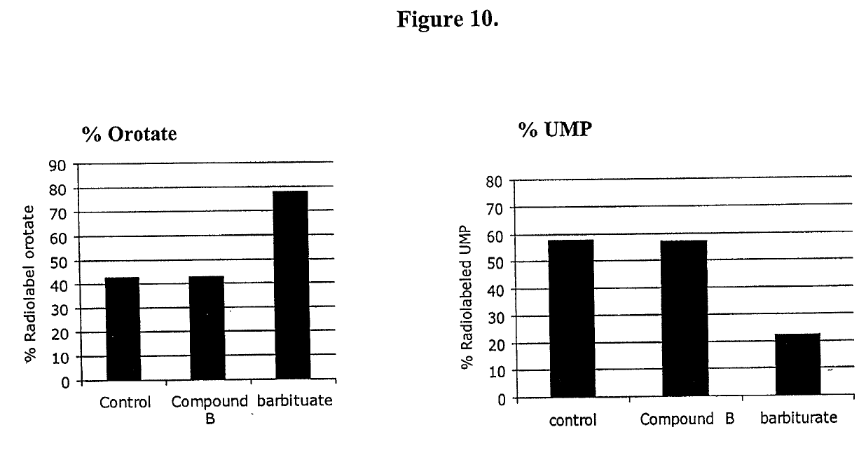

When [¹⁴C]-orotate was used as a substrate, enzyme activity was high and stable in a desalted crude extract. Within 60 minutes after treatment, all substrate ([¹⁴C]-orotate) was converted to UMP (FIG. 8). The conversion of [¹⁴C]orotate into UMP was dependent on PRPP (FIG. 9). To our surprise, probe compound did not inhibit the in vitro incorporation of [¹⁴C]-rotate to UMP compared to control (FIG. 10). In contrast, a known inhibitor (barbiturate) inhibited the conversion of [¹⁴C]-orotate to UMP by 80%.

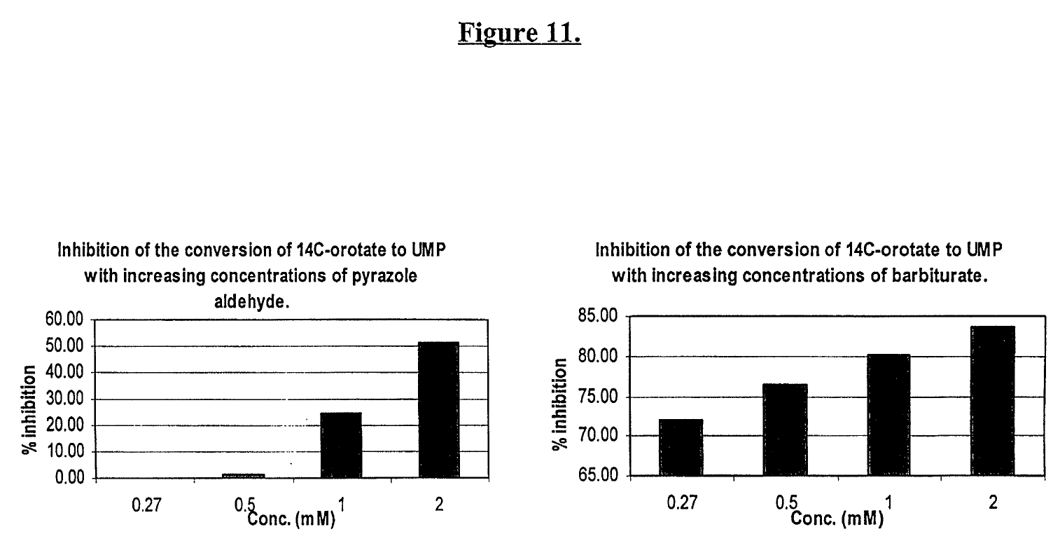

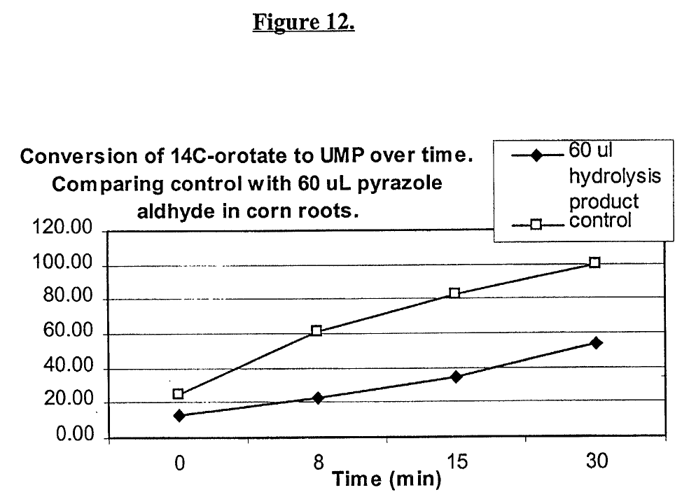

It was later discovered that the probe compounds hydrolyze in solution and the breakdown product inhibits the enzymatic conversion of [¹⁴C]-orotate to UMP (FIG. 11). The estimated IC₅₀ for the inhibition of OPRTase/ODCase by the breakdown product of the probe compound was 2 mM, which was 100 times higher than the IQo for probe compound whole plant injury detected in the miniscreen and in the in vivo incorporation of [¹⁴C]-carbamoyl aspartate into uracil/uridine. FIG. 12 illustrates that the inhibition by the breakdown product of the probe compound increases over time.

The potency of the breakdown product of the probe compound may be affected by further metabolism such as phosphorylation. For example, 6-azaurdine is a potent inhibitor only after being phosphorylated to 6-azauridine-mono-phosphate (Saenger, W. et al. (1973) Nature 242:610-612). 6-Azauridine was tested in our enzyme assay, and was not shown to inhibit OPRTase/ODCase activity, suggesting that the enzymes or components necessary for phosphorylation of this compound in vivo were not functional in our cell free extract. Therefore, the present invention encompasses the phosphorylation of a compound identified by the method of the present invention. The compound may be metabolically, enzymatically, and/or synthetically phosphorylated using methods known in the art. Alternatively, in the instance where the compound requires phosphorylation for biological activity, the present invention encompasses the de-phosphorylation of the compound as a means of inhibiting the activity of the compound.

Dihydroorotase/Dihydroorotate Dehydrogenase (DHO/DHOD).

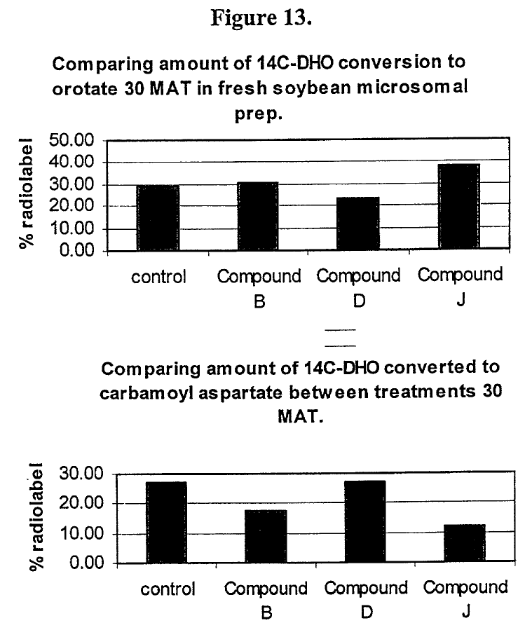

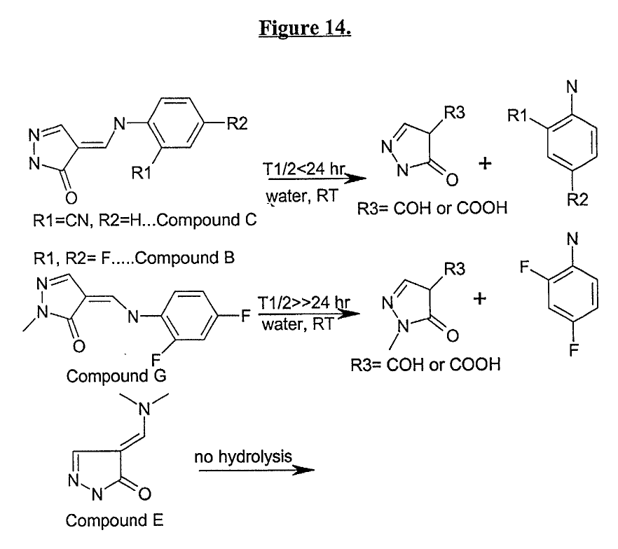

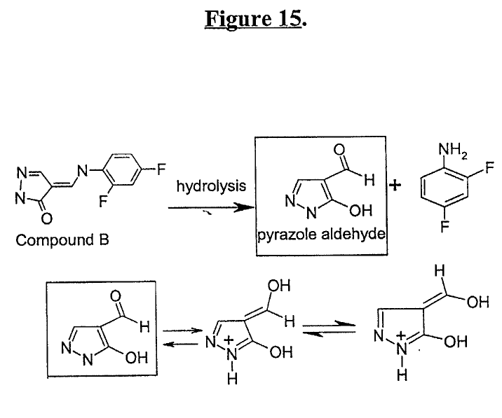

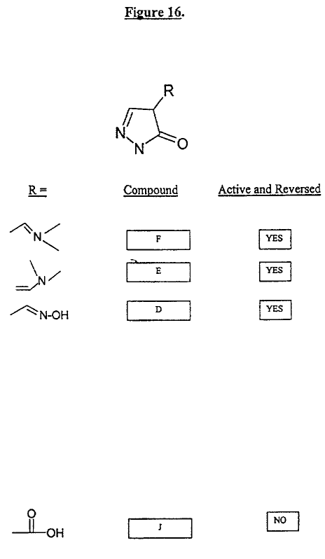

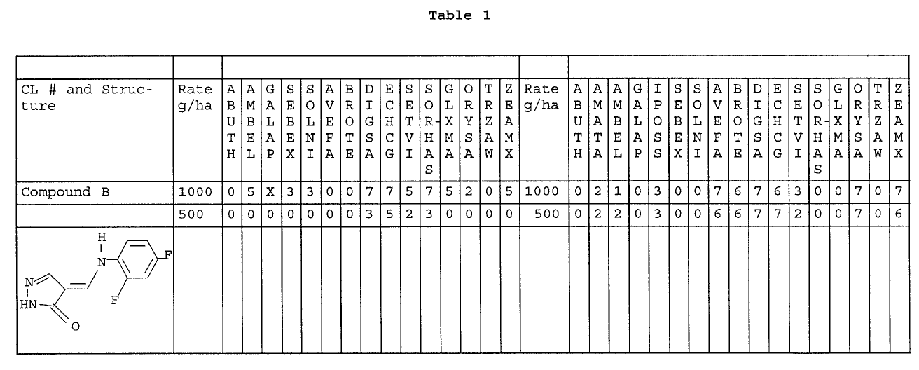

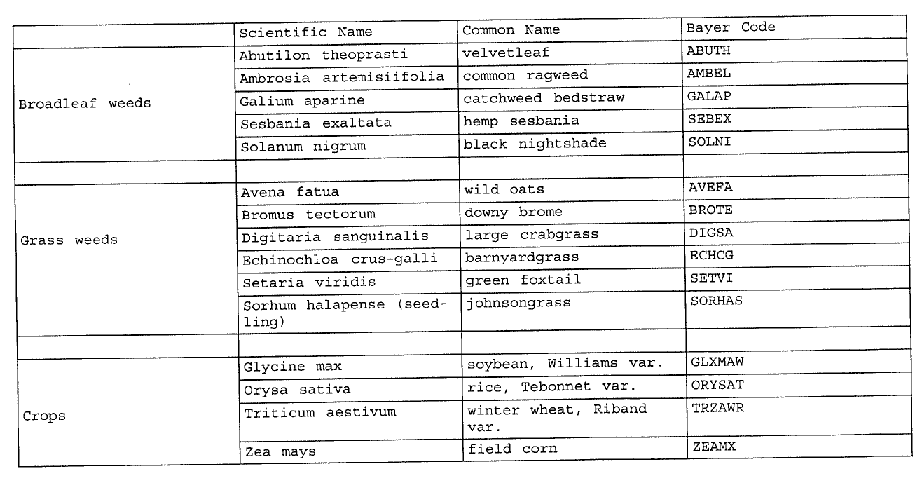

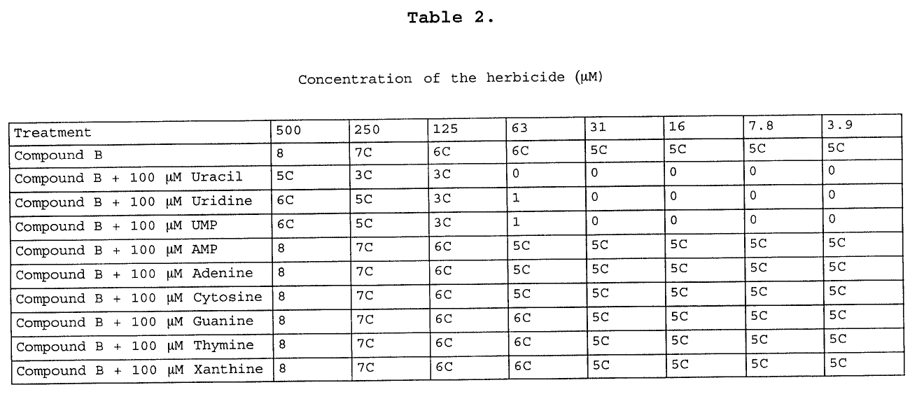

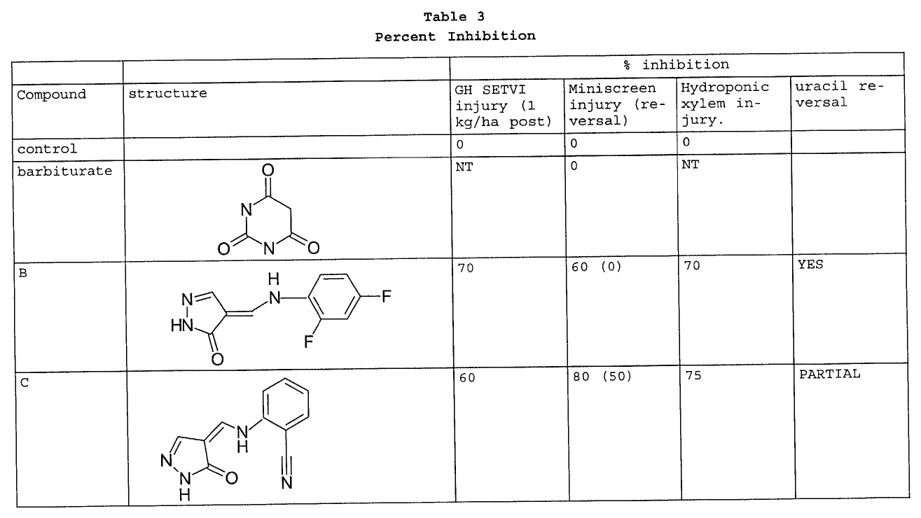

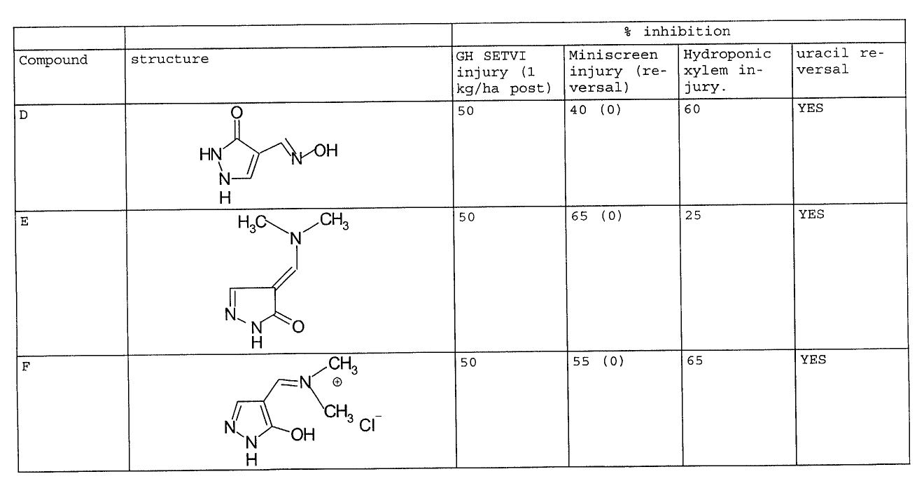

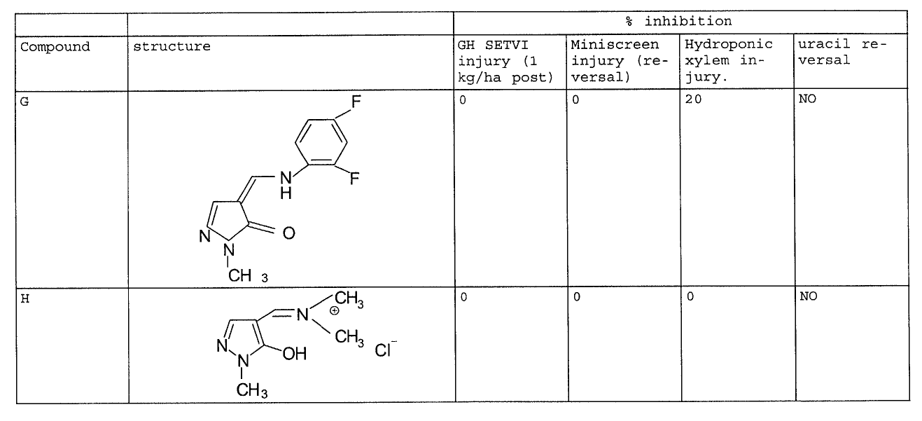

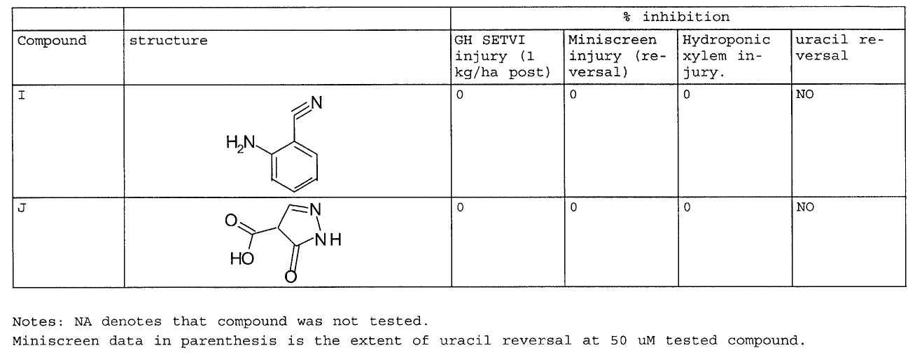

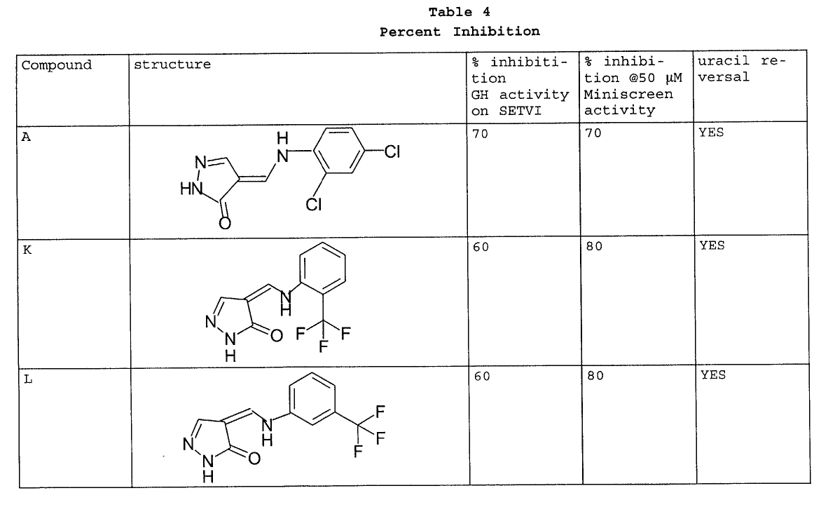

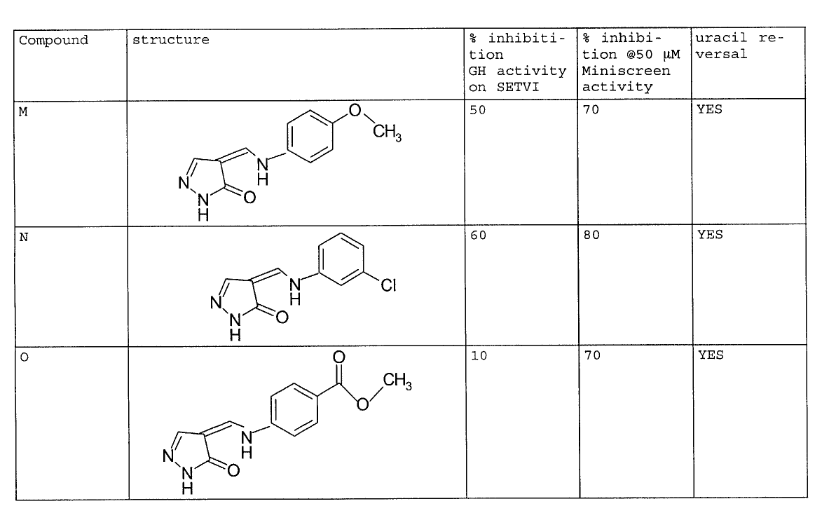

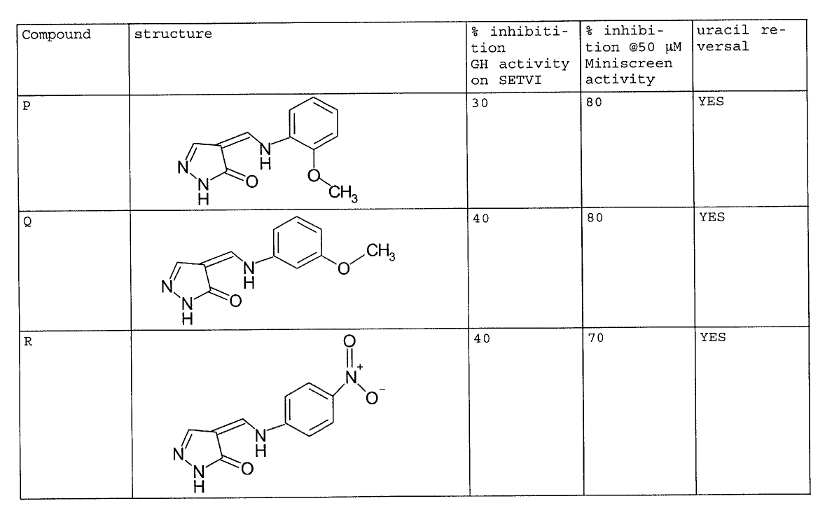

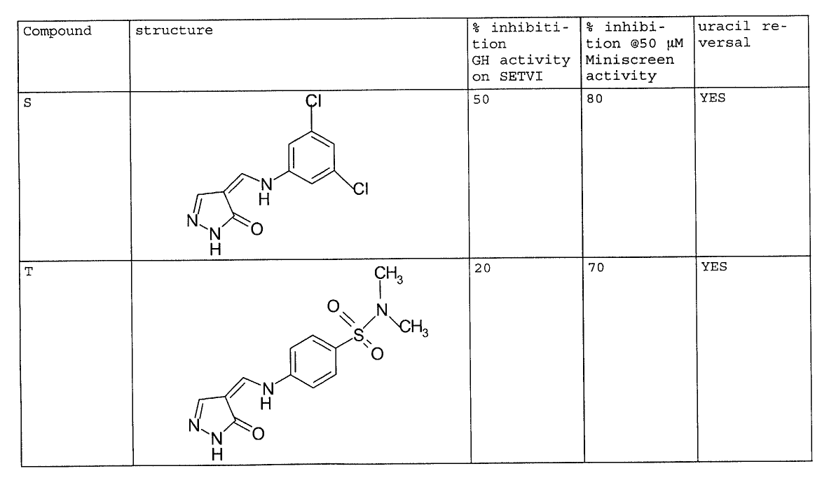

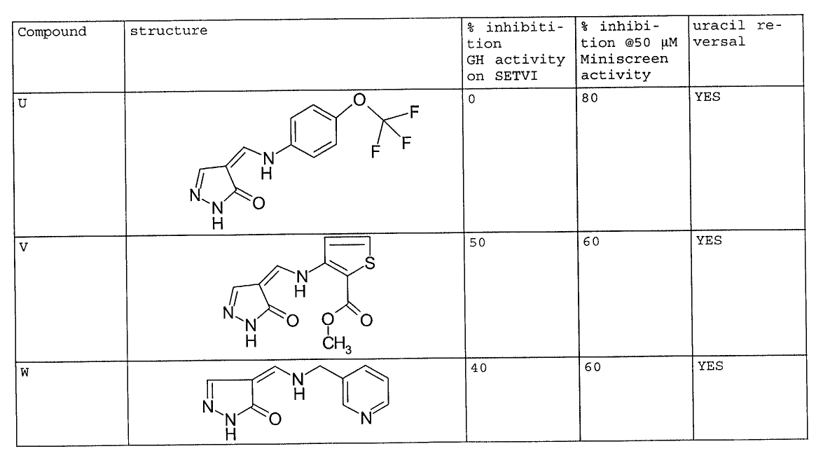

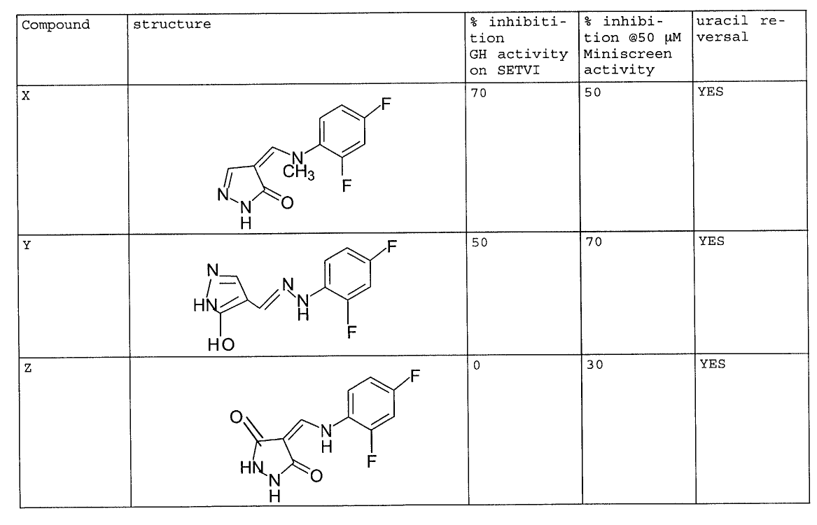

As shown in FIG. 13, the probe compound did not inhibit the activity of either DHO or DHOD. The assays were performed using [¹⁴C]dihydroorotate as a substrate and monitoring its conversion to orotate (DHOD activity) or the reverse reaction from [¹⁴C]dihydroorotate to carbamoyl aspartate. These results suggest that the probe compound does not inhibit DHO or DHOD activity under these particular assay conditions.

Methods:

Etiolated corn roots and shoots or soybean suspension cells were used as tissue. Corn seedlings were germinated on vermiculite for 4 days in the dark at 26_C. and roots and shoots harvested separately. The soybean cells were grown in media consisting of Muashige and Skoog salts (Physiol. Plant (1962) 15:473-497) purchased from Gibco/BRL, vitamins (glycine 2 mg/L, thiamine 0.1 mg/L, nicotinic acid 0.5 mg/L, pyridoxine-HCl 0.5 mg/L), myo-inositol (100 mg/L), 2,4-D (0.4 g/L), and 30 g/L sucrose. Cell cultures are grown at 25° C. with gentle shaking. Cultures were transfered every 7 days to new growth media.

Approximately 5-10 g plant tissue were ground in liquid nitrogen and resuspended in 1:2 (w/v) homogenization buffer (30 mM KPO₄⁻; 25 mM KCl₂, 5 mM MgCl₂, 5 mM ascorbic acid, pH 7.2). The homogenate was centrifuged at 10,000 g for 20 min, and the supernatant desalted using BioRad columns. The desalted supernatant was used for OPRTase/ODCase enzyme assays. For DHOD enzyme extraction, the supernatant was further centrifuged at 100,000 g for 45 min to obtain a microsomal pellet. The microsomal pellet was resuspended in 3-4 ml resuspension buffer (70 mM Tris-base, pH 7.4) containing 1% Triton X-100.

All enzyme assays were performed according to Rawls, J. M. (1978) Anal. Biochem. 86 (1): 107-117 and Cleaveland et al. (1995). Biochem. Pharm. 49(7):947-954. Enzyme assays were performed in a total volume of 200 ul with 1 uM [¹⁴C]carbamoyl aspartate, [¹⁴C]dihydroorotate or [¹⁴C]orotate as substrates, and 1-5 mM probe compound or barbiturate as inhibitors. The [¹⁴C]carbamoyl aspartate and [¹⁴C]dihydroorotate assay was performed using a potassium phosphate buffer, pH 7.4 with 12 mM NAD⁺, and 10 mM MgCl₂. The [¹⁴C]orotate enzyme assay was performed using Tris buffer, pH 8.8 with 12 mM PRPP and 10 mM MgCl₂. Over time, 25 ul aliquots were taken from the reaction tube and boiled for 5 min to stop the reaction. Samples were spotted onto cellulose TLC plates and run using 0.1 M potassium phosphate buffer, 100% ammonium sulfate, and 1-propanol (10:6:1). Plates were developed using a TLC plate radioisotope detector. Orotate and UMP standards were run next to unknowns to identify the conversion of [¹⁴C]orotate to [¹⁴C]UMP in the enzyme assay.

### Example 6

### Stability of the Lead Uracil Biosynthesis Inhibitors, Compound “B”, Compound “C”, Compound “G”, and Compound “E”

As referenced in Example 5, several of the lead probe compounds that demonstrate uracil biosynthesis inhibitory activity, readily hydrolyze in solution. Using a combination of mass spectroscopy and NMR analysis, it was determined that the compound “B” and compound “C” compounds have half-lives less than 24 hours in aqueous solution. Upon hydrolysis, the compounds are believed to breakdown into their respective anilines, in addition to either a pyrazole aldehyde or pyrazole acid (See FIG. 14). However, the results indicate the presence of various isomers in the hydrolyzed solution—clearly suggesting the presence of various tautomeric forms and possibly other species.

Although the pyrazole aldehyde was separately isolated and subjected to the enzyme assays of the present invention, the uracil biosynthesis inhibitory activity of this breakdown species was significantly than barbiturate, but higher than the lead compound (e.g., compound “B”). The latter result suggests that the pyrazole aldehyde may not be the active species of the lead compound. However, the activity of the pyrazole aldehyde may be dependent upon additional processing (e.g., phosphorylation, reduction, oxidation, etc.). Alternatively, the compound “B” parent compound may, in fact, be the active species and the observed breakdown reaction may not be relevant to the compounds uracil biosynthesis inhibitory activity. Similarly, it is possible that the compound “B” compound may be processed enzymatically or chemically, in the cell (e.g., phosphorylation, reduction, oxidation, etc.). Such processing may stabilize the compound, or alternatively, may convert the compound into the active species. In addition, the lower uracil biosynthesis inhibitory activity of the pyrazole aldehyde species may be due to decreased cellular uptake of this compound with respect to the parent compound, which may explain why higher concentrations of the breakdown species is required to inhibit uracil biosynthesis.

In contrast to the above compounds, the compound “G” compound was found to be relatively stable in aqueous solution with a half-life significantly greater than 24 hours. Although hydrolysis of this compound into its respective aniline was observed (in addition to the pyrazole aldehyde or acid), it is significant that this compound demonstrated only marginal uracil biosynthesis inhibition (see Table III). Thus, suggesting that either the stereochemistry of the aniline nitrogen is significant to the uracil biosynthesis inhibitory activity of the intact compound, or that the active species of the compound is, in fact, a hydrolysis product of the compound that is not observed as readily due to the decreased rate of hydrolysis.

The compound “E” compound was determined to be stable in aqueous solution with no hydrolysis detected. Contrary to the compound “G” compound, this compound demonstrated significant uracil biosynthesis inhibitory activity. Since this compound is very similar in structure to the pyrazole aldehyde hydrolysis product of the compounds compound “B” and compound “C”, the latter result may provide additional evidence that the pyrazole aldehyde, or isomers thereof, is the active species.

Methods:

The stability analysis of the compound “B”, compound “C”, compound “G”, and compound “E” compounds was performed using a Hewlett Packard HP1100 LC system coupled to a Micromass Platfom-LC mass spectrometer operating with an electrospray ionization source. In positive ion electrospray, AC_{12254 82}A produced molecules at m/z 112(M⁺), m/z 153 (M+41) at a cone voltage of 30V. Retention time of AC 12254 82A was 12.54 min with absorption maxima of 248 and 314 nm.

LC Conditions

Column: Hypersil Spherisorb Waters ODS(2), 3.6×150 mm

Eluent: 60% acetonitrile, 40% water (0.1% formic acid)

Flow rate: 150 μL/min

Injection: 5 μg (5 ul)

Photodiode Array Detection: Scan Range: 190-600 nm

1H NMR Conditions:

1H NMR was taken in DMSO-d₆ on a 500 MHZ Bruker NMR Spectrometer. The analysis showed the existence of two species in equilibrium. Each had 5 protons as follows: NH, 2OH and 2 CH.

### Example 7

### Preparation of (5Hydroxy-1H-pyrazol-4-ylmethylene)-dimethylammonium Chloride

A cold solution of 21 g 1,2-dihydropyrazol-3-one (0.125 mol) in 50 ml anhydrous dimethylformamide was treated with 40 g phosphorous oxythiochloride (0.15 mol) over 1 h period at a temperature at or below 20° C. The reaction mixture was warmed to room temperature and then heated to 90-95° C. and held for 1 h. The thick slurry was cooled in an ice bath and diluted with 80 ml absolute ethanol keeping the temperature below 0° C. After stirring for 4 h, the product crystallized in white solids. After filtration and drying in a vacuum, the product of melting point 234° C. was obtained in 76.5% yield.

### Example 8

### Preparation of (5-Hydroxy-1-methyl-1H-pyrazol4-ylmethylene)-dimethylammonium Chloride

A cold solution of 4.9 g 2-methyl-1,2-dihydropyrazol-3-one (0.05 mol) in 10 ml anhydrous dimethylformamide was treated with 9.2 g phosphorous oxythiochloride (0.06 mol) over 1 h period at a temperature at or below 10° C. The reaction mixture was heated to 90-95° C. and held for 1 h. The thick slurry was cooled and diluted with 20 ml absolute ethanol keeping the temperature below 0° C. After filtration and drying in a vacuum, the product was obtained in 68% yield and characterized by NMR spectra.

### Example 9

### Preparation of 4-[[(2,4-difluorophenyl)amino]methylene]-2,4-dihydro-3H-pyrazol-3-one and Tautomers Thereof

A slurry of 1.75 g (5-Hydroxy-1H-pyrazol-4-ylmethylene)-dimethylammonium chloride (0.01 mol) in 15 ml absolute Ethanol was warmed to 70° C., treated with 1.3 g 2,4-difluoroaniline (0.01 mol), then heated for 5-10 min to 80° C. and cooled afterwards. The emerging precipitating product (yellow solid) was obtained in 71% yield and characterized afterwards by ¹H and ¹³C NMR. The product was shown to be a mixture of tautomers in solution by NMR, one maijor isomer (MA) and one minor isomer (MI) (see sheme 3).

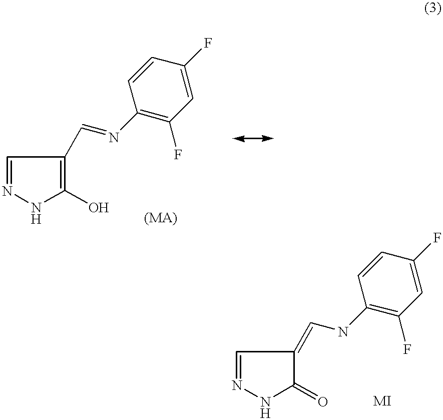

¹H NMR of the major isomer in DMSO-□₆: [symbol] \tilde{11} □ [symbol] (N1-H); 8.63 (vinyl H), 7.76 (J_{H-F}=5.5 Hz, J_{H-H}=9.22 Hz, aromatic 6′H), 7.54 (pyrazole 3H), 7.49 (J_{H-F}=10.11 Hz, J_{H-H}=2.82 Hz, aromatic 3′H), 7.23 (J_{H-F}=10.11 and 1.54 Hz, J_{H-H}=9.22 and 2.82 Hz, aromatic 5′H), (OH not seen)

¹³CNMR of the major isomer in DMSO-□₆: [symbol] 167.9 (C5), 158.6 (J_{C-F}=244.5 and 11.6 Hz, aromatic C4′), 152.1 (J_{C-F}=247.2 and 12.6 Hz, aromatic C2′), 145.5 (vinyl C), 124.1 (J_{C-F}=9.2 Hz, aromatic C1′), 118.5 (J_{C-F}=9.3 Hz, aromatic C6′), 112.4 (J_{C-F}=22.7 and 3.5 Hz, aromatic C5′), 104.9 (J_{C-F}=27.3 and 23.1 Hz, aromatic C3′), 102.1 (pyrazole C4)

Majorpeaks in ¹HNMR in DMSO-□₆: [symbol] 10.96 (N1-H); 10.75 (NH), 7.95 (pyrazol 3H), 7.65 (vinyl H)

### Example 10

### Preparation of 4-[[2-cyanophenyl)amino]methylene]-2,4-dihydro-3,4-pyrazol-3-one and Tautomers Thereof

A solution of 0.88 g (5-Hydroxy-1H-pyrazol-4-ylmethylene)-dimethylammonium chloride (0.05 mol) in 10 ml absolute Ethanol at 50° C. was treated with 0.59 g 2-aminobenzonitrile (0.009 mol), incubated for 10 to 15 min and cooled afterwards. After drying the product (yellow solid) was obtained in 32% yield and characterized by 1H, 13C NMR spectroscopy and was shown to be a mixture of tautomers in solution like (Example 9).

### Example 11

### Preparation of 4-[[(2,4-difluorophenyl)amino]methylene]-2,4-dihydro-2-methyl-3H-pyrazol-3-one and Tautomers Thereof

A slurry of 1.9 g (5-Hydroxy-1-methyl-1H-pyrazol-4-ylmethylene)-dimethylammonium chloride (0.01 mol) in 15 ml absolute Ethanol at 70° C. was treated with 1.3 g 2,4-difluoroaniline (0.01 mol) and incubated for 10 to 15 min at 80° C. and cooled afterwards. After drying the product (yellow solid) of melting point 180° C. was obtained in 73% yield and characterized by ¹H NMR spectroscopy.

¹HNMR of the major isomer in DMSO-□₆: [symbol] 8.65 (vinyl H), 7.77 (J_{H-F}=5.5 Hz, J_{H-H}=9.22 Hz, aromatic 6′H), 7.58 (pyrazole 3H), 7.49 (J_{H-F}=10.11 Hz, J_{H-H}=2.82 Hz, aromatic 3′H), 7.23 (J_{H-F}=10.11 and 1.54 Hz, J_{H-H}=9.22 and 2.82 Hz, aromatic 5′H), 3.23 (N-methyl) (OH not seen)

### Example 12

### Preparation of 4-[[(2,4-difluorophenyl)methylamino]methylene]-2,4-dihydro-2-methyl-3H-pyrazol-3-one and Isomers Thereof

A slurry of 0.88 g (5-Hydroxy-1H-pyrazol-4-ylmethylene)-dimethylammonium chloride (0.005 mol) in 10 ml absolute Ethanol at 70° C. was treated with 0.12 g N-methyl-2,4-difluoroaniline (0.005 mol) and incubated for 20 min at 80° C. and cooled afterwards. After drying the product (yellow solid) of melting point 181-181.5° C. was obtained in 42% yield.

Throughout this application, various publications are referenced, the disclosures of which are hereby incorporated by reference in their entireties into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for identifying compounds that inhibit uracil biosynthesis comprising the following steps:
i) applying test compounds to plant tissue in the presence of pyrimidine biosynthetic pathway intermediates or end-products;
ii) applying test compounds to plant tissue in the absence of pyrimidine biosynthetic pathway intermediates or end-products;
iii) comparing the effects of the test compound from step i) with the effects of the test compounds from step ii); and
iv) noting the test compounds in step iii) that show reversed effects in the presence of said intermediates or end-products.

2. The method according to claim 1, wherein the intermediates or end-products are selected from the group consisting of: uracil, uridin-5′-monophosphate, uridine, orotate, OMP, aspartate, carbamoyl phosphate, N-carbamoyloaspartate, L-dihydroorotate, NAD+, PRPP, UDP, UTP, cytidine, and thymidine.

3. The method according to claim, 1 further comprising the step of determining which test compounds have herbicidal effects.

4. The method according to claim 1, further comprising the step of determining which test compounds have fungicidal, insecticidal or nematicide effects.

5. An herbicidally active compound having the formula I: 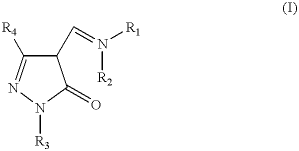
wherein:
R₁ is hydrogen or C₁-C₆-alkyl unsubstituted or substitued with one to three of the following radicals: cyano, nitro, hydroxyl, mercapto, amino, carboxyl, C₁-C₄-alkoxy, C₁-C4-haloalkoxy, C₁-C₄-alkoxycarbonyl or C₁-C₆-alkylthio;
R₂ is hydroxy, OR₅, aryl, arylmethylene, hetaryl, hetarylmethylene unsubstituted or substitued with one to three of the following groups: halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, and C₁-C₆-alkoxycarbonyl;
R₃ is C₁-C₄-alkyl or hydrogen;
R₄ is hydrogen or hydroxy; and
R₅ is C₁-C₆-alkylcarbonyl or a formyl-moiety bonded to the structure via the carbon atom;
with the proviso that if R₁ is H, R₂ can also be HNR₆, wherein R₆ is aryl, arylmethylene, hetaryl, hetarylmethylene unsubstituted or substituted with halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, or C₁-C₆-alkoxycarbonyl;
or an agriculturally acceptable salt or ester thereof.

6. The herbicidal compound of claim 5, wherein the compound is:
4-[[(2,4-dichlorophenyl)amino]methylene]-2,4-dihydro-3H-pyrazol-3-one;
4-[[(2,4-difluorophenyl)amino]methylene]-2,4-dihydro-3H-pyrazol-3-one;
2-[[(5-oxo-1,5-dihydro-4H-pyrazol-4-ylidene)methyl]amino]benzonitrile;
2,3-dihydro-3-oxo-1H-pyrazole-4-carboxaldehyde-4-oxime;
4-[(dimethylamino)methylene]-2,4-dihydro-3H-pyrazol-3-one;
Hydrochloride of 4-[(dimethylamino)methylene]-2,4-dihydro-3H-pyrazol-3-one;
4-[[(2,4-difluorophenyl)amino]methylene]-2,4-dihydro-2-methyl-3H-pyrazol-3-one,;
Hydrochloride of 4-[(dimethylamino)methylene]-2,4-dihydro-2-methyl-3H-pyrazol-3-one;
2-aminobenzonitrile, 4,5-dihydro-5-oxo-1H-pyrazole4-carboxylic acid;
4-[[[2-trifluoromethyl)phenyl]amino],methylene]-2,4-dihydro-3H-pyrazol-3-one;
4-[[[3-(trifluoromethyl)phenyl]amino],methylene]-2,4-dihydro-3H-pyrazol-3-one;
4-[[(4-methoxyphenyl)amino]methylene]-2,4-dihydro-3H-pyrazol-3-one;
4-[[(3-chlorophenyl)amino]methylene]-2,4-dihydro-3H-pyrazol-3-one;
4-[[(1,5-dihydro-5-oxo-4H-pyrazol-4-ylidene)methyl]amino]benzoic acid, methyl ester;
4-[[(2-methoxyphenyl)amino]methylene]-2,4-dihydro-3H-pyrazol-3-one;
4-[[(3-methoxyphenyl)amino]methylene]-2,4-dihydro-3H-pyrazol-3-one;
4-[[(4-nitrophenyl)amino]methylene]-2,4-dihydro-3H-pyrazol-3-one;
4-[[(3,5-dichlorophenyl)amino]methylene]-2,4-dihydro-3H-pyrazol-3-one;
4-[[(1,5-dihydro-5-oxo-4H-pyrazol-4-ylidene)methyl]amino]-N,N-dimethylbenzenesulfonamide;
4-[[[4-(trifluoromethoxy)phenyl]amino], methylene]-2,4-dihydro-3H-pyrazol-3-one;
3-[[(1,5-dihydro-5-oxo-4H-pyrazol-4-ylidene)methyl]amino]-2-thiophenecarboxylic acid, methyl ester;
4-[[(3-pyridylmethyl)amino]methylene]-2,4-dihydro-3H-pyrazol-3-one;
4-[[(2,4-difluorophenyl)methylamino]methylene]-2,4-dihydro-3H-pyrazol-3-one;
5-hydroxy-1H-pyrazole4-carboxaldehyde, 2,4-difluorophenylhydrazone; or
4-[[(2,4-difluorophenyl)amino]methylene]-3,5-pyrazolidinedione.

7. An herbicidal composition comprising the herbicidal compound of claim 5 and a carrier.

8. A method of herbicidally treating an undesired plant comprising contacting the undesired plant or area intended for crop plants with an herbicidally effective amount of the compound of claim 5.

9. A method of herbicidally treating an undesired plant comprising contacting the undesired plant or area intended for crop plants with an herbicidally effective amount of the compound of composition of claim 7.

10. A process for preparing the compound of claim 5, wherein R4 is hydrogen, comprising reacting an amoniumsalt of the formula II 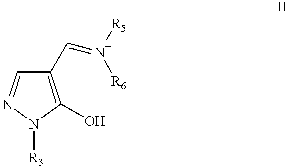
wherein R₃ is hydrogen or C₁-C₄-alkyl, and R₅ and R₆ are both independently hydrogen or C₁-C₄-alkyl or both together for C₃-C₅ alkylene; with an amine of formula III 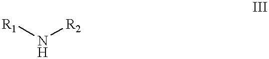
wherein
R₁ is hydrogen or C₁-C₆-alkyl unsubstituted or substitued with one to three of the following radicals: cyano, nitro, hydroxyl, mercapto, amino, carboxyl, C₁-C₄-alkoxy, C₁-C₄-haloalkoxy, C₁-C₄-alkoxycarbonyl or C₁-C₆-alkylthio; and
R₂ is hydroxy, OR₅, aryl, arylmethylene, hetaryl, hetarylmethylene unsubstituted or substitued with one to three of the following groups: halogen, cyano, nitro, hydroxyl, mercapto, amino, carboxyl, aminocarbonyl, aminothiocarbonyl, C₁-C₆-alkyl, C₁-C₆-haloalkyl, C₁-C₆-alkoxy, C₁-C₆-haloalkoxy, and C₁-C₆-alkoxycarbonyl;
to produce the compound of claim 5.

11. A process for preparing the compound of the formula II of claim 10 comprising reacting a pyrazolone of the formula IV 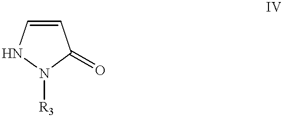
wherein R₃ is hydrogen or C₁-C₄-alkyl in Vilsmeyer reaction with a dialkylformamide of formula V 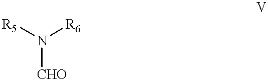
wherein R₅ and R₆ are both independently hydrogen or C₁-C₄-alkyl or both together for C₃-C₅ alkylene; to produce the compound of formula II.

12. A compound of the formula II as defined in claim 10, or an agriculturally salt or ester thereof.

13. A process for production of pesticidal agents comprising the following steps:
a) identification of the uracil biosynthesis inhibitor comprising
i) applying test compounds to plant tissue in the presence of pyrimidine biosynthetic pathway intermediates or end-products;
ii) applying test compounds to plant tissue in the absence of pyrimidine biosynthetic pathway intermediates or end-products;
iii) comparing the effects of the test compound from step i) with the effects of the test compounds from step ii); and
iv) noting the test compounds in step iii) that show reversed effects in the presence of said intermediates or end-products; and
b) preparing a pesticidal composition comprising the pesticidal agent identified in step a).

14. A method for identifying the mode of action of a probe compound comprising:
i) treating a plant with one or more radiolabeled pyrimidine biosynthesis intermediates in the presence of probe compound,
ii) treating a plant with one or more radiolabeled pyrimidine biosynthesis intermediates in the absence of probe compound,
iii) determining the percent conversion of said radiolabeled pyrimidine biosynthesis intermediates to radiolabeled pyrimidine biosynthesis products selected from the group consisting of: orotate, uridine-5′-monophosphate, and uracil,
iv) comparing the percent conversion of said intermediates to said products of “ii” with those from step “iii”, and
v) noting specific accumulations of said products in the presence of probe compound as compared to the absence of probe compound.

15. The method according to claim 14, wherein the plant is a member selected for the group consisting of: seeds, seedlings, germinated seeds, emerging seedlings, plant tissue, meristematic tissue, root tissue, stem tissue, flower tissue, cotyledon tissue, shoot tissue, callus, plant cultures, plant cells, plant vegetation, plant roots, Arabidopsis plants, and Arabidopsis seeds.

16. The method according to claim 15, wherein the radiolabeled pyrimidine biosynthesis intermediates are selected from the group consisting of: [14C] carbamoyl aspartate, [14C] dihydroorotate, [14C] orotate, [14C]-orotidine-5′-monophosphate, [14C]-uridine-5′-monophosphate, [14C]-uridine-5′-diphosphate, [14C]-uridine-5′-triphosphate.
